(19) United States
(12) Patent Application Publication
(10) Pub. No.: US 2017/0151446 A1
(43) Pub. Date: Jun. 1, 2017

(54) METHOD AND APPARATUS FOR EFFECTING ALTERNATING ULTRASONIC TRANSMISSIONS WITHOUT CAVITATION

(71) Applicant: BKR IP HOLDCO LLC, Broomall, PA (US)

(72) Inventor: Bruce K. REDDING, JR., Broomall, PA (US)

(73) Assignee: BKR IP Holdco LLC, Broomall, PA (US)

(21) Appl. No.: 15/323,604

(22) PCT Filed: Jul. 6, 2015
(86) PCT No.: PCT/US2015/039251

§ 371 Date: Jan. 3, 2017

Related U.S. Application Data
(60) Provisional application No. 61/998,623, filed on Jul. 3, 2014.
(60) Provisional application No. 61/998,622, filed on Jul. 3, 2014.
(60) Provisional application No. 61/998,624, filed on Jul. 3, 2014.
(60) Provisional application No. 61/998,683, filed on Jul. 7, 2014.
(60) Provisional application No. 61/998,788, filed on Jul. 9, 2014.
(60) Provisional application No. 61/998,790, filed on Jul. 9, 2014.
(60) Provisional application No. 61/999,589, filed on Aug. 1, 2014.
(60) Provisional application No. 62/124,758, filed on Jan. 2, 2015.
(60) Provisional application No. 62/125,836, filed on Feb. 2, 2015.
(60) Provisional application No. 62/125,837, filed on Feb. 2, 2015.

(51) Int. Cl. A61N 7/00

(52) U.S. Cl. CPC A61N 7/00; A61N 2007/0073

(57) ABSTRACT

Ultrasound generation produces in general an acoustic field, characterized by both inertial and non-inertial acoustic cavitation, a process by which non-linear oscillation of a microbubble and its associated micro streaming and radiation force generated by ultrasound can lead to intense heating effects in a material, solution or biological cell which comes into contact with a conventional ultrasound transmission. Typically an ultrasound signal contains both an acoustic vibration effect, a resonance effect where a material receiving the ultrasound transmission resonates in response to the transmission, and unfortunately in many applications a damaging cavitation effect and a damaging thermal effect. This invention is both a method and an apparatus to reduce the damaging effects of ultrasound in both the thermal and mechanical effects and to provide a safer ultrasonic process which can be used in sonochemistry applications, material science and for biological or medical applications.

### PRIORITY CLAIM, CROSS REFERENCE TO RELATED APPLICATIONS AND INCORPORATION BY REFERENCE

This application is related to, claims priority under, and claims the benefit of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790; “METHOD AND APPARATUS FOR MEASURING THE DOSE REMAINING UPON A TRANSDERMAL DRUG DELIVERY DEVICE”, Bruce K. Redding, Jr., filed on Aug. 1, 2014, and having Ser. No. 61/999,589; “ULTRASONICALLY ENHANCED SEED GERMINATION SYSTEM SOIL TREATMENT PROCESS”, Bruce K. Redding, Jr., filed on Jan. 2, 2015, and having Ser. No. 62/124,758; “ULTRASONIC TREATMENT OF SEEDS DELTA SEED MACHINE”, Bruce K. Redding, Jr., filed on Feb. 2, 2015, and having Ser. No. 62/125,836; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837.

This application hereby incorporates herein by reference the subject matter disclosed in the abstracts, the written descriptions, the drawings, and the claims, in their entireties, of the following provisional applications filed in the United States Patent and Trademark Office: “MODIFIED TRANSDERMAL DELIVERY PATCH WITH MULTIPLE ABSORBENT PADS”, Bruce K. Redding, Jr., filed on Jul. 3, 2014, and having Ser. No. 61/998,623: “MODIFIED TRANSDERMAL DELIVERY DEVICE OR PATCH AND METHOD OF DELIVERING INSULIN FROM SAID MODIFIED TRANSDERMAL DELIVERY DEVICE”, Bruce K. Redding, Jr., filed on Jul. 3, 2014, and having Ser. No. 61/998,622; “METHOD FOR GLUCOSE CONTROL IN DIABETICS”, Bruce K. Redding, Jr., filed on Jul. 3, 2014, and having Ser. No. 61/998,624; “ULTRASONIC TRANSDUCERS SUITABLE FOR ULTRASONIC DRUG DELIVERY VIA A SYSTEM WHICH IS PORTABLE AND WEARABLE BY THE PATIENT”, Bruce K. Redding, Jr., filed on Jul. 7, 2014, and having Ser. No. 61/998,683; “METHOD FOR THE ATTENUATION ENHANCEMENT OF ABSORBENT MATERIALS USED IN BOTH PASSIVE AND ACTIVE TRANSDERMAL DRUG DELIVERY SYSTEMS”, Bruce K. Redding, Jr., filed on Jul. 9, 2014, and having Ser. No. 61/998,788; “MODIFICATION OF PHARMACEUTICAL PREPARATIONS TO MAKE THEM MORE CONDUCIVE TO ULTRASONIC TRANSDERMAL DELIVERY”, Bruce K. Redding, Jr., filed on Jul. 9, 2014, and having Ser. No. 61/998,790; “METHOD AND APPARATUS FOR MEASURING THE DOSE REMAINING UPON A TRANSDERMAL DRUG DELIVERY DEVICE”, Bruce K. Redding, Jr., filed on Aug. 1, 2014, and having Ser. No. 61/999,589; “ULTRASONICALLY ENHANCED SEED GERMINATION SYSTEM SOIL TREATMENT PROCESS”, Bruce K. Redding, Jr., filed on Jan. 2, 2015, and having Ser. No. 62/124,758; “ULTRASONIC TREATMENT OF SEEDS DELTA SEED MACHINE”, Bruce K. Redding, Jr., filed on Feb. 2, 2015, and having Ser. No. 62/125,836; “METHOD AND APPARATUS FOR EFFECTING ALTERNATING ULTRASONIC TRANSMISSIONS WITHOUT CAVITATION”, Bruce K. Redding, Jr., filed on Feb. 2, 2015, and having Ser. No. 62/125,837.”

### BACKGROUND OF THE INVENTION

The present invention relates to a method and an apparatus for reducing or eliminating the cavitation forces in an acoustic transmission while retaining the vibratory energy associated with said acoustic transmission. The invention also relates to non-cavitation ultrasound generating systems.

One aspect of the present invention relates to an ultrasonic device which produces reduced or no cavitation forces, or temperature effects as a result of alternating the waveform of the sonic transmission.

Reference is made to the following publications:
The Temperature of Cavitation, Edward B. Flint and Kenneth S. Suslick, Science, New series, Volume 253, Issue 5026 (Sep. 20, 1991), 1397-1399; and “Ultrasound, Cavitation bubbles and their interaction with Cells”, Junru Wu and Wesley L. Nyborg, Elsevier, Advanced Drug Delivery Review 60 (2008) 1103-1116, Apr. 8, 2008.

A review of the referenced material indicates that ultrasound is generally formed in a single waveform, a sine wave, as indicated in FIG. 1. A standard acoustical transmission in the ultrasonic range, above 20 kHz frequency, possesses a positive part of the wave front called the compression wave which is followed by a negative portion of the wave front, termed the expansion section.

FIG. 1 shows the positive and negative portions of a typical sine wave transmission. The quick drop off of pressure is shown in the graph of FIG. 4 and can lead to intense cavitation effects as disclosed in the Suslick article.

Referring to FIG. 1 again, it can be seen that a typical sinusoidal ultrasonic transmission grows to an implosive effect typically at the 400 micro-second (msecs) duration point in the transmission. Once at 400 msecs, the sinusoidal ultrasound transmission has formed a bubble in the solution or substrate exposed to the ultrasonic transmission with as high as a 150 micron radius and then generates a shock wave which causes the bubble to collapse. In FIG. 1, the microject shockwave collapses the bubble and in the process of collapse, a hot spot or instantaneous temperature rise occurs at the microscopic level in the solution or substrate exposed to the ultrasonic transmission.

Suslick describes a cavitation temperature range as high as 5075+/−156° K within 1 millionth of a second.

This intense cavitation effect and the resultant temperature rise can have the effect of damaging materials, biological structures or cells and denaturing pharmaceutical preparations as indicated in FIG. 3.

A further review of the fundamental physics of ultrasonic waves indicates non-focused ultrasonic plane-traveling waves as shown in FIG. 35, a plane-wave sound source and its wave front.

Wu and Nyborg disclose that ultrasound within a fluid or within a biological tissue can have cavitation effects: considering a half-space (x>0) filled with a liquid or soft tissue. For most cases, the soft-tissue may be considered as a liquid like medium. At x=0, there is a thin solid-plane as shown in FIG. 35 with lateral dimensions (perpendicular to x direction) much greater than the wavelength (λ) of the sound wave. This plane, a sound source, is vibrating sinusoidally with time and back and forth in space around its initial position at x=0; its vibration leads to the production of a sound wave in the region x>0. The displacement of this source plane with respect to x=0 can be written as (Eq-1):

x(t)=A cos(2πft+φ₀),

where f is frequency of the vibration, A (>0) is the amplitude, φ₀ is the initial phase which determines the initial (t=0) conditions of the source plane. For example, if φ₀=0, the displacement and velocity of the source plane are x=A and v=dx/dt=0, respectively, when t=0. In the regime of linear acoustics (discussion of non-linear acoustics in later sections), a traveling pressure wave propagating along x direction in a medium is generated by the vibrating sound source.

That is to say, the pressure in the medium is a function of x and t and fluctuates around the atmospheric pressure. If we define the acoustic pressure p(x, t) as the excess of the total pressure to the atmospheric pressure, it can be written as (Eq-2):

p(x,t)=P₀(x)cos(kx−ωt)=p₀e^{-αx} cos(kx₀−ωt),

where P₀(x) is the acoustic pressure amplitude which is a function of position x and is equal to

p₀e^{-αx}, where p₀=P₀ (0), the pressure amplitude at x=0. Other parameters include the angular frequency ω=2πf, the propagation constant (or wave number) k=2π/λ and the attenuation coefficient of the medium α.

In water, α is approximately a linear increasing function of frequency in the megahertz range.

The attenuation coefficient α describes the energy transfer from the sound wave to the medium mainly through absorption and scattering processes. Absorption converts acoustic energy irreversibly into heat mainly via viscous friction. Inside the tissue or in aqueous suspensions of cells, inhomogeneities exist.

Scattering is a process whereby the inhomogeneities re-direct some sonic energy to regions outside the original wave-propagation path. If the density of the inhomogeneity is high, multiple-scattering may occur. In other words, in such instances sonic energy may scatter among several inhomogeneities back and forth for several times before it is diminished by absorption. In water, the attenuation coefficient α is often negligible and the multiplying factor e^{-αx} may be considered to be unity in Eq. (2).

Frequency and wavelength are not independent for a sound wave; they are related by the relationship of fλ=c, where c is called the phase velocity. In water, the phase velocity at 20° C. is approximately equal to 1500 m/s.

Noting that if x=x₀, p(x, t) in Eq. (2) becomes

p(x,t)=P₀(x)cos(kx₀−ωt)=p₀e^{-αx}₀ cos(kx₀−ωt);

thus, the acoustic pressure at any point on the plane x=x₀ changes sinusoidally in time, the phase being equal to kx₀−ωt. A plane or a surface where every point has the same phase is called a wavefront. An acoustic wave which has a set of planes as its wavefronts and can be represented by Eq. (2) is often called a non-focused plane-traveling wave. When the frequency f is above the typical human audible range (f≧20 kHz), this type of sound wave is called ultrasound (US). In principle, the plane wavefront of a traveling wave described by Eq. (2) has infinite dimensions. In practice, however, a simple sound source is often a circular ceramic disk that exhibits a piezoelectric effect and has a radius a of a finite dimension; it is also called a “piston” sound source. The nature of the US generated by the piston source is quite different from a plane-traveling wave; it depends on the ratio α/λ. However, under the condition a [symbol] λ, the sound wave in the far-field region (which will be defined later) behaves like an ultrasonic beam with a circular cross section. Within the beam, particularly close to the beam axis, the acoustic pressure may be approximately described by Eq. (2).

### Interaction Between Ultrasound and Bubble Formation and Cavities:

Basic physics of free bubbles and microbubbles lead to “acoustic cavitation” which refers to activities associated with air or gas bubbles, pockets and cavities under excitations of acoustic waves. There are two types of bubbles related to the sonoporation application: free bubbles and encapsulated microbubbles (EMB), as shown in FIG. 2. Free bubbles are usually cavities filled with air, other gases, or gas vapor from surrounding liquid. Unlike EMBs, they have no artificial boundaries to prevent leakage of air or gas from the bubbles. They are not stable in a liquid for a variety of reasons. They may float to the top of the liquid and disappear under the influence of gravitational force or may be dissolved into the liquid because of the so-called “Laplace pressure imbalance” due to the surface tension or they may coalesce into larger bubbles. Microscopic free bubbles may be stably present in cracks or other irregularities on solid surfaces or on small dust particles or impurities. Those microscopic bubbles may grow in size as the time of the ultrasonic transmission lengthens as shown in FIG. 1.

Therefore the formation of cavitation is tied to the waveform dynamic of the ultrasound

Inertial (Transient) and Non-Inertial (Stable) Cavitation. Transmission.

There are two types of acoustic cavitation: (1)“inertial” and (2) “non-inertial”. Inertial cavitation, formerly called “transient” cavitation, occurs if the acoustic pressure amplitude is sufficiently high and above a threshold level. Under this condition the EMBs will first grow in volume, and then implode violently.

If the core of an EMB is gas of high κ(=Cp/Cv), high temperature may result during implosions and highly reactive free radicals may be generated. For some biological and other effects, inertial cavitation seems to be required and for others it should be avoided.

Non-inertial cavitation, formerly called “stable” cavitation; occurs when an EMB in a liquid is forced to oscillate with only a relatively small to moderate increase and decrease of radius as shown in FIG. 2 (off-resonance regime), when the pressure amplitude of the external acoustic field is not too high.

Acoustic microstreaming and shear stress associated with the waveform and ultrasound propagation in liquids or biological tissue is a non-linear partial differential equation. In general, the propagation speed of a traveling plane wave in a medium is a function of particle velocity of the medium.

If the amplitude of ultrasound becomes significantly large (many applications in diagnostic and therapeutic ultrasound applications belong to that category), the linear approximation does not hold any more; leading to acoustic streaming—a steady and direct current (DC) flow in an acoustic field can result again in bubble formation and collapse and intense thermal effects. One of the acoustic streaming phenomena relevant to sonoporation is microstreaming, which leads to repeat implosion, shockwave and hot spot formation in a liquid, followed by rapid quenching and then a cycling back to shockwave growth upon the recycle.

The vibratory effects of ultrasound are welcome in many industrial, chemical, biological and drug delivery applications, however the cavitation effect can damage the material under ultrasonic transmission and can result in thermal effects which detract from the overall vibratory effects.

It is a purpose of this invention to provide a method and an apparatus for obtaining ultrasonic vibration, with reduced cavitation or thermal effects. This is accomplished by disrupting the following factors in the ultrasonic transmission:

(1) Disrupt the timing sequence of the ultrasonic transmission (UT) by reducing the transmission time below 400 msecs. FIG. 1 shows that for typical sinuosdial ultrasound, which is the current waveform dynamic associated with ultrasound that below 400 msecs the formation of an implosion-shockwave-hot spot thermal effect can be minimized. The optimum cavitation avoidance is to drop the cycling below 400 msecs. Arbitrarily, the use of a 100 msec cycle instead of a 400 msec cycle was chosen in the apparatus described below, however the cycle could have been 200 or 300 or some other variation below 400 msecs. Other non-limiting examples include varying the ultrasound timing below 400 msecs, for instance from about 50 msecs to about 90 msecs for the first waveform and from about 10 msecs to about 50 msecs for the second waveform, such as 80 msces for the first, leading waveform and 20 msces for the second, following waveform, or other variations which include 70/30, or 90/10 respective msecs for the first leading waveform and the second, following waveform.

(2) Conventional ultrasound is limited to sinusoidal waveforms because that is the limit of the transducer. Conventional transducers emit sine wave based waveforms as shown in FIG. 13, which shows that no matter the waveform of the electrical signal delivered to the transducer, the mechanical force emits as a sinusoidal waveform. To provide a cavitation-free ultrasonic transmission, the transducer design needed to be revised to allow for a match between the electronic signal delivered to the transducers and the resultant mechanical waveform, as shown in FIG. 14.

(3) A further means of providing a cavitation-free ultrasonic transmission is to alternate the waveform. In FIG. 1 the variation of acoustic pressure, between the compression positive and the expansion negative, leads eventually to an implosion, shockwave, thermal effect, i.e. cavitation. Within the normal 400 msec time period there is a build up to the cavitation effect. By disrupting that buildup pattern the cavitation ultrasonic transmission can be minimized or forced to not form in the first cycle and in subsequent acoustic cycles. The use of an alternating waveform dynamic instead of a sinusoidal waveform can be used to disrupt cavitation formation. FIG. 5 shows the use of an alternating signal to drop off the cavitation growth, as shown in FIG. 1, through the use of at least two waveforms, waveform A which is a different waveform dynamic than waveform B. In FIG. 5 waveform A can be a sawtooth waveform, which has a timing function below 100 msecs, and ideally 50 msecs. Just before any semblance of a cavitation growth pattern, as shown in FIG. 1, can form, the waveform A converts to waveform B, a totally different wave dynamic. In FIG. 5 waveform B is a square waveform. Referring back to FIG. 1, this alternating waveform (from one form, such as sawtooth or sine to another, such as square) interrupts the formation of cavitation and eliminates the cavitation growth track in the ultrasonic transmission

FIG. 5 shows a cavitation free ultrasonic transmission which relies on 4 components:
Component 1: A priming sequence of one waveform, such as sawtooth, shown for a period of just 30 msecs, which can be used to prime the material, chemical agent or biological structure to ultrasound. In drug delivery the sawtooth waveform is used to dilate the pores of the skin as shown in FIG. 28.
Component 2: The waveform “A” transmission.
Component 3: The waveform “B” transmission, which should be a different waveform than the waveform “A” transmission.

A null gap between the waveforms to relax the ultrasonic transmission, and thereby avoid cavitation further.

Various combinations can be used to affect the alternating waveform dynamic including: FIG. 6: (A) sine to sawtooth; (B) FIG. 7: sine to square (C) FIG. 8: sawtooth to square; (D) FIG. 10: triangular to square. Any combination of alternating waveforms can be used to minimize cavitation ultrasound. A transducer according to the present invention is capable of delivering mechanically a waveform fed to it electronically from a first waveform to any other second waveform, wherein the waveforms are any one of a sine waveform, a sawtooth waveform, a square waveform and a triangular waveform. Preferably the first and second waveforms are different.

(4) An alternate method to minimize cavitation ultrasound is to use a waveform transmission which automatically loses energy during the transmission stage, and thereby never reaches the implosion, shockwave and hot spot effect normally associated with sinusoidal ultrasound. The use of a triangular waveform dynamic as shown in FIG. 9 where the waveform slides through a frequency range, leading to a drop in the amplitude, can also be used to avoid the cavitation formation.

(5) In FIG. 11 the cavitation drop off is effected by switching the ultrasonic waveform by a change in either the Duty Cycle or the Timing Cycle associated with the ultrasonic propagation. In FIG. 11 the Duty Cycle is varied so that the waveform switches every so many milliseconds. In FIG. 12 the Timing cycle is altered so that the alternating wave dynamic is deactivated in a gap period of time before the alternating waveform recycles. That gap period is a totally deactivated signal, which again stops the growth pattern first shown in FIG. 1 and stops cavitation from forming.

Other variations of the use of alternating or combination waveforms may be employed to avoid cavitation ultrasound and the inventor does not want to be limited by the combinations illustrated herein.

These and other objects of the invention can be accomplished by applying various ultrasound frequencies, intensities, amplitudes and/or phase modulations to control the magnitude of the transdermal flux to achieve a cavitation free ultrasonic transmission, using the vibratory effects of the ultrasound to accomplish the purpose of the directed no-cavitation ultrasound.

### BRIEF SUMMARY OF THE INVENTION

One aspect of the invention is the use of phase modulation, alternating waveforms, timing cycles and frequency modulation to achieve more effective ultrasonic transmissions, which exhibit little or no cavitation or thermal effects.

Another aspect of the invention is a method of providing cavitation free ultrasound in an ultrasonic device, whereby an ultrasonic signal employs a combination of two or more waveforms, and whereby the growth of the acoustic signal is interrupted from becoming cavitational.

Another aspect of the invention is the combination of alternating waveforms, to effect cavitation free ultrasound, via an ultrasonic transmission device, a transducer which will propagate mechanically the electronic waveform delivered to it.

Still another aspect of the invention is a transducer which is capable of delivering mechanically a waveform fed to it electronically from a first waveform to any other second waveform, wherein the waveforms are any one of a sine waveform, a sawtooth waveform, a square waveform and a triangular waveform.

Yet another aspect of the invention is a transducer which is capable of delivering cavitation free ultrasound, which employs a reflector on a top face of the transducer to reflect ultrasonic signals back to a target.

Another aspect of the invention is a transducer which is capable of delivering cavitation free ultrasound, which employs one or more individual transducer discs or elements in an array, placed over a stainless steel face plate, and which cause the face plate to irradiate harmonic ultrasound in resonance to the ultrasound delivered from the transducer discs affixed to it, wherein the face plate and transducer disc array are covered by a block containing a flexible foam rubber layer between the stainless steel face plate and the block housing, whereby increasing overall intensity of the transducer and increasing the diameter of surface area to the overall sonic transmission.

Still another aspect of the invention is a method of delivering cavitation free ultrasound, which produces a sonic pattern upon a target, which is spherical and which avoids troughs in the beam profile, thereby avoiding cavitation effects upon the target material subjected to the ultrasound transmission.

Another aspect of the invention is a method of delivering cavitation free ultrasound, which employs one or more alternating sonic waveforms where one waveform is a triangular wave front where the frequency and amplitude of the wave front is diminishing over time, thereby preventing the growth of a cavitation or thermal effect to the ultrasound transmission.

Additional objects, advantages and novel features of the invention will be set forth in part in the description which follows, and in part will become apparent to those skilled in the art upon examination of the following, or may be learned by practice of the invention.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown.

In the drawings:

FIG. 1 is an illustration of the effects of conventional ultrasound in the formation of an implosion, shockwave and thermal effect which leads to cavitation.

FIG. 2 is an illustration of bubble formation and collapse as a result of cavitation ultrasound.

FIG. 3 is an illustration of drug degradation which can be effected via cavitation ultrasound.

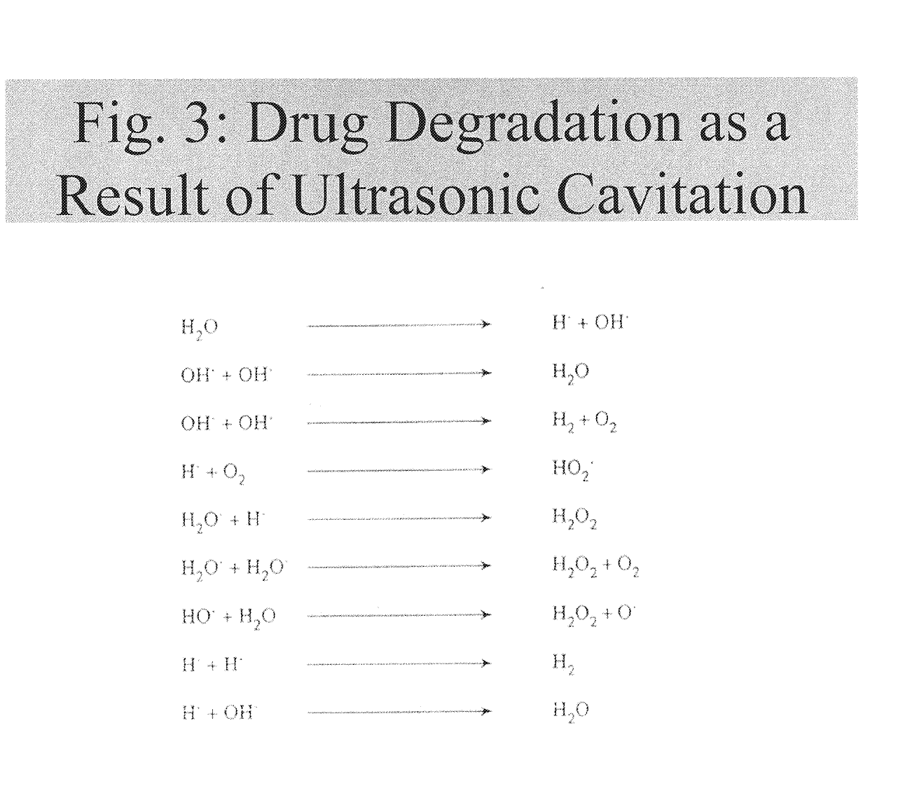

FIG. 4 shows the compression and expansion effects of cavitation ultrasound.

FIG. 5 shows the cavitation drop off that can be effected through the use of an alternating waveform dynamic wherein a waveform A is followed by a different wave structure in the waveform B transmission.

FIG. 6 is a combination of sine to sawtooth waveform.

FIG. 7 is a combination of sine to square waveform.

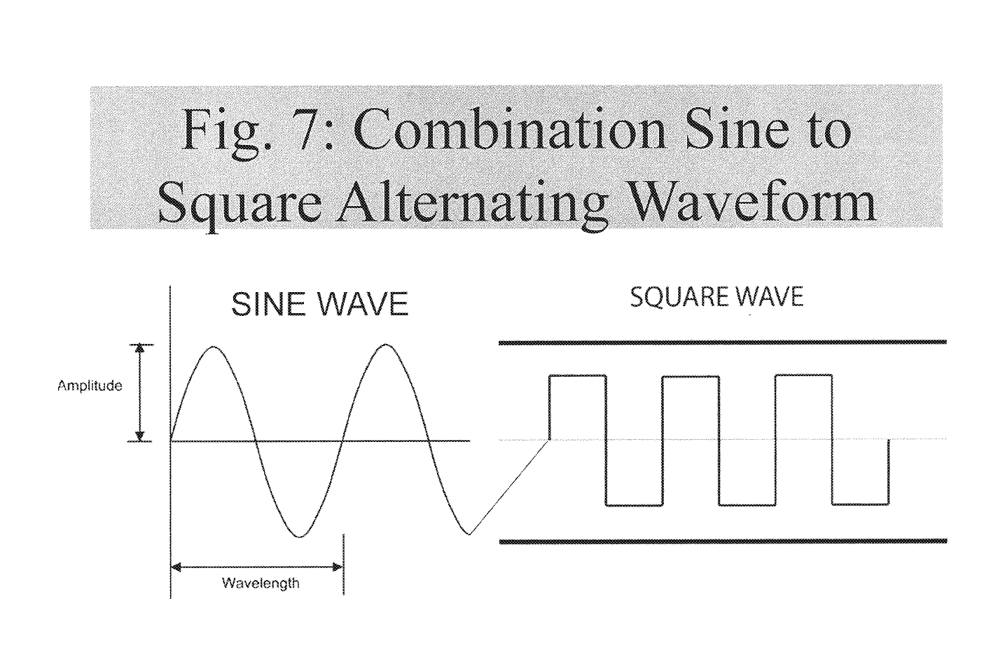

FIG. 8 is a combination of sawtooth to square waveform.

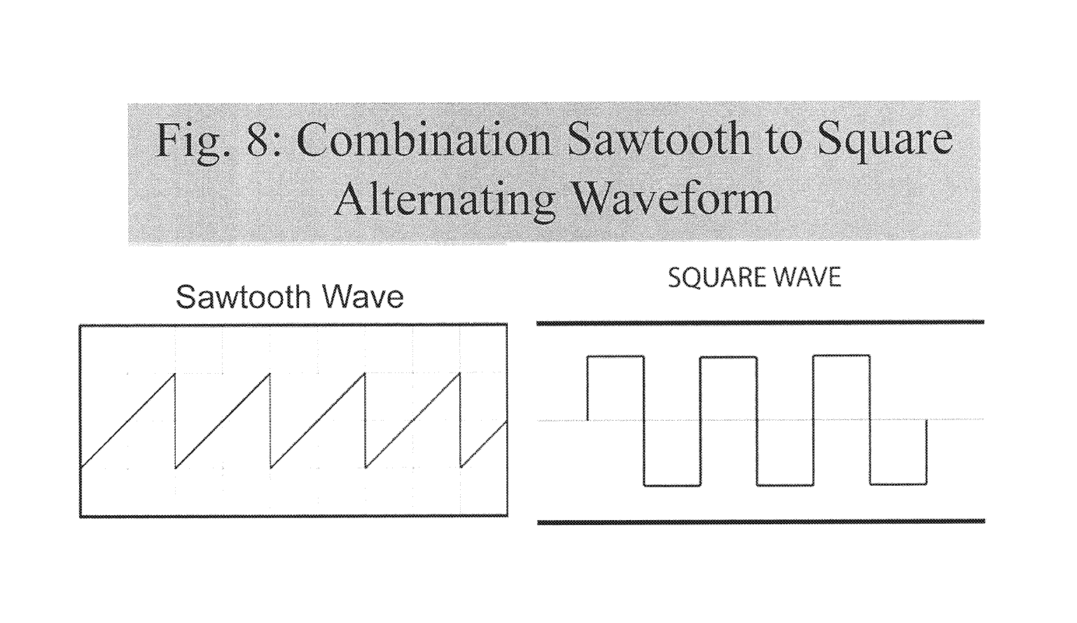

FIG. 9 is an illustration of the use of a triangular waveform dynamic, where the waveform slides through a frequency range, leading to a drop in amplitude, to avoid the cavitation formation.

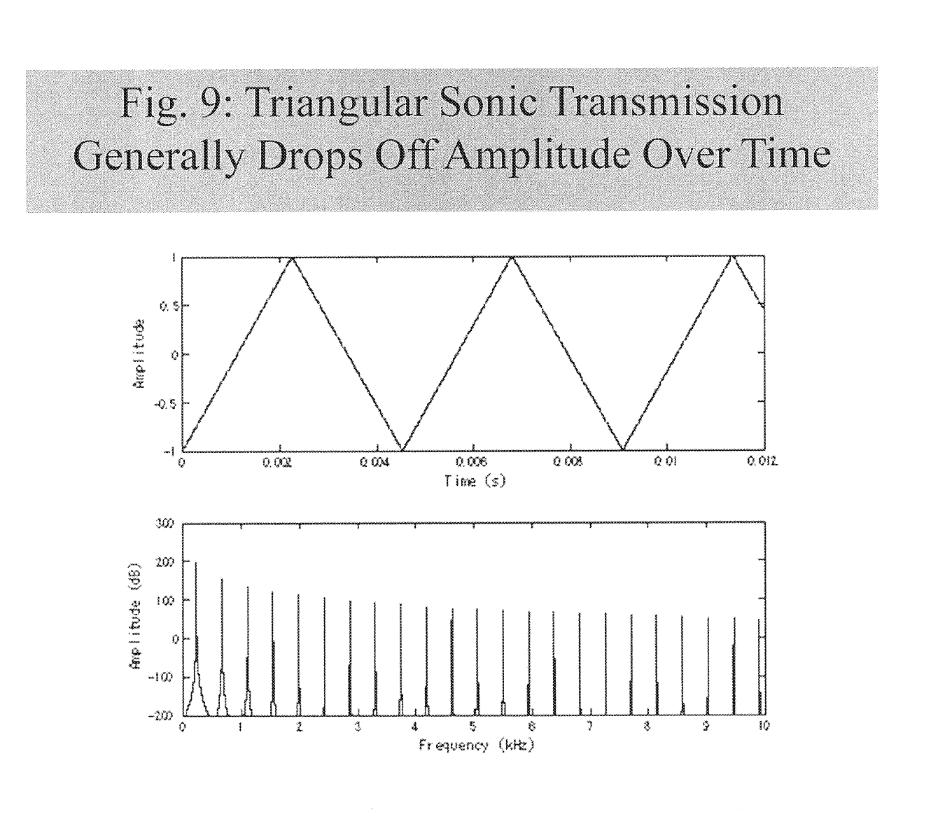

FIG. 10 is a combination of triangular to square waveform combination.

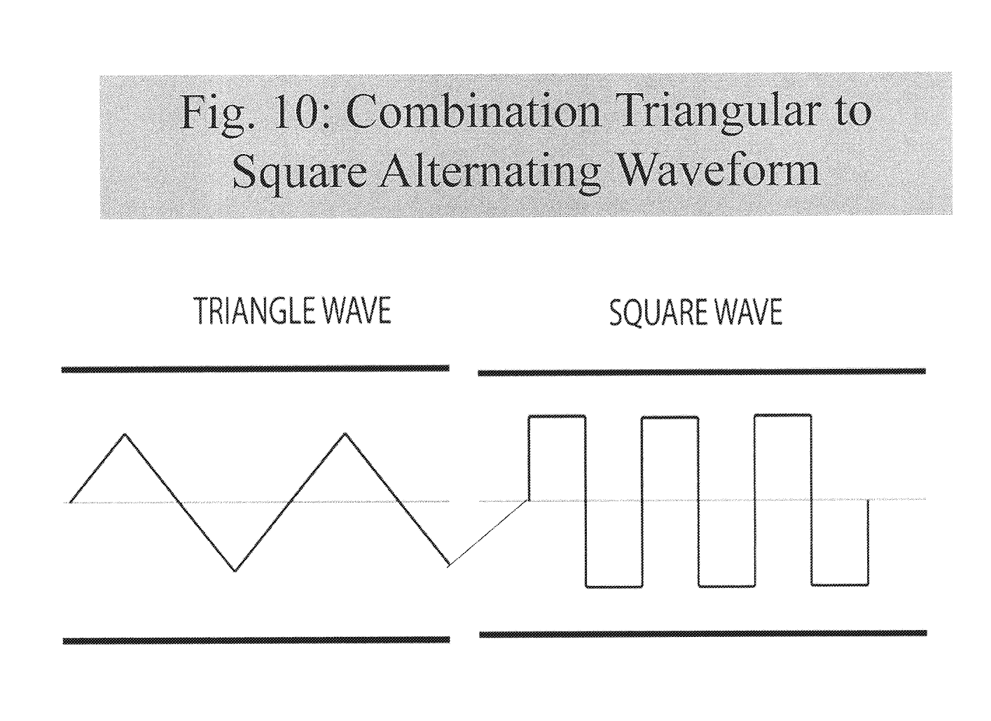

FIG. 11 illustrates a cavitation drop off by switching the ultrasonic waveform timing cycle.

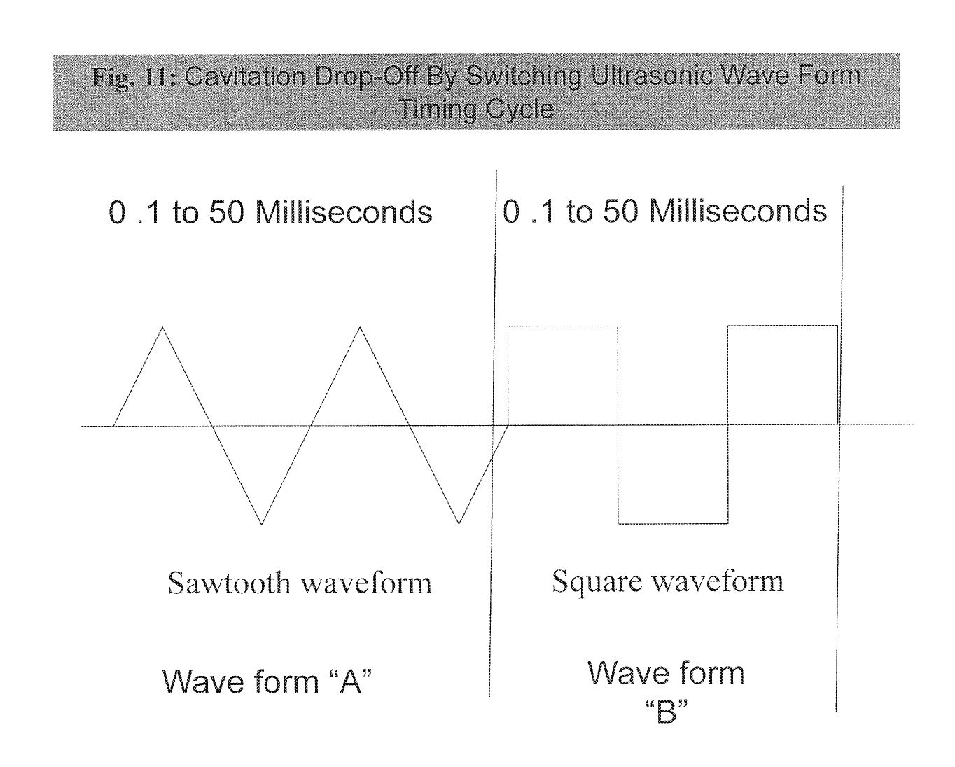

FIG. 12 illustrates the use of a timing cycle to interrupt the formation of cavitation.

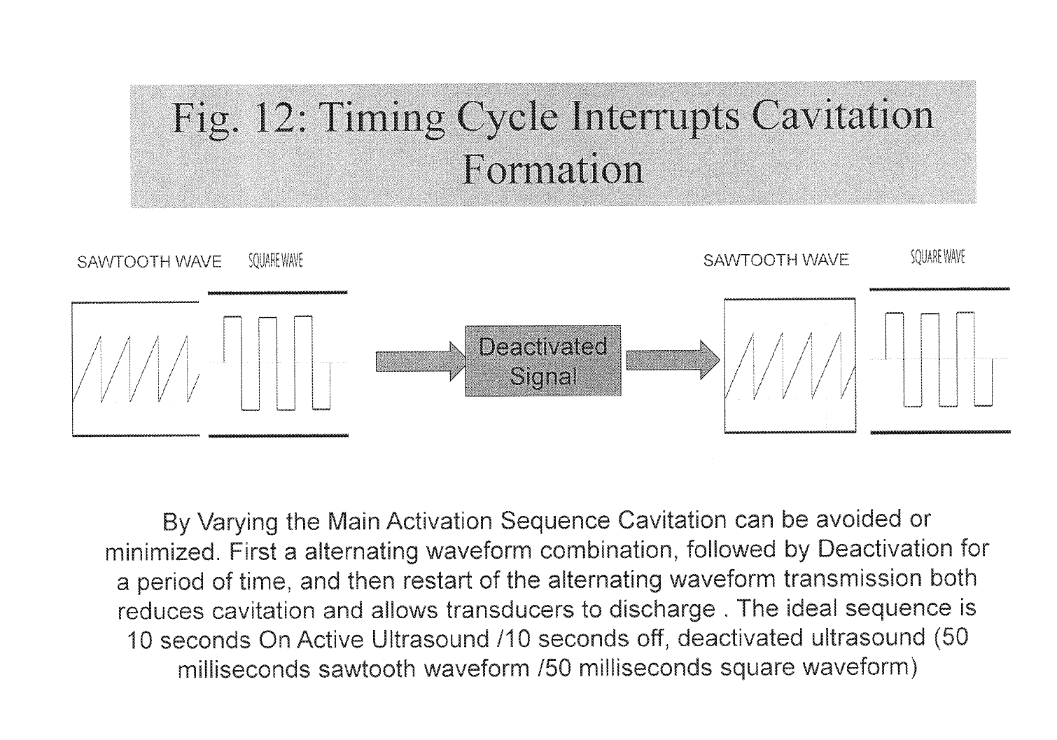

FIG. 13 shows that no matter the waveform of the electrical signal delivered to the transducer, the mechanical force emits as a sinusoidal waveform.

FIG. 14 shows the transducer of the new design which utilizes a special transducer construction which will provide a cavitation-free ultrasonic transmission, by allowing for a match between the electronic signal delivered to the transducers and the resultant mechanical waveform.

FIG. 15 is a modified transducer design utilizing a reflector design to focus the ultrasound toward the target.

FIG. 16 shows the reflective transducer which minimizes “loose” ultrasound transmission and focuses the ultrasonic signal in one direction.

FIG. 17 is a modified transducer design utilizing a reflector design to focus the ultrasound toward the target, and shows the placement pattern of transducer discs upon a face plate.

FIG. 18 shows binary or stacked transducer configurations.

FIG. 19 shows a “C-type single element transducer disc.

FIG. 20 shows the transducer reflector casing.

FIG. 21 shows a 9-element transducer array.

FIG. 22 shows a 4-element transducer array.

FIG. 23 is an assembly diagram showing the formation of a transducer block, capable of delivering no-cavitation ultrasonic transmissions.

FIG. 24 illustrates the test apparatus used in Experiments 1A and 1B.

FIG. 25 illustrates the result of Experiment 2.

FIG. 26A illustrates the results of Experiment 3 upon insulin compared to regular insulin. FIG. 26A is an HPLC Spectra of Lispro Insulin, A control sample which has not been subject to ultrasound.

FIG. 26B is the HPLC Spectra of Lispro Insulin, which has been subject to the alternating ultrasound transmission of 50 milliseconds sawtooth, followed by 50 milliseconds square wave ultrasound, showing no damage occurred to the insulin sample.

FIG. 27 shows the damage caused by conventional low power ultrasound upon insulin.

FIG. 28 shows pore dilation effects upon the skin using sawtooth waveform ultrasound directed against the skin.

FIG. 29 is an acoustic pattern in water of a single element transducer producing an alternating waveform.

FIG. 30 is a beam profile comparison of alternating ultrasonic transmission at 25 kHz and 40 kHz.

FIG. 31 is a beam profile comparison of conventional sinusoidal cavitation ultrasonic transmission at 25 kHz and 40 KHz.

FIG. 32 is a beam profile comparison of conventional cavitation ultrasound transmission at 60 KHz.

FIG. 33 is a beam profile comparison of conventional cavitation ultrasound transmission at 80 kHz and 100 KHz.

FIG. 34 is a circuit diagram of the electronic alternating ultrasonic generator used to effect cavitation free ultrasound.

FIG. 35 shows a plane-wave sound source and its wavefront

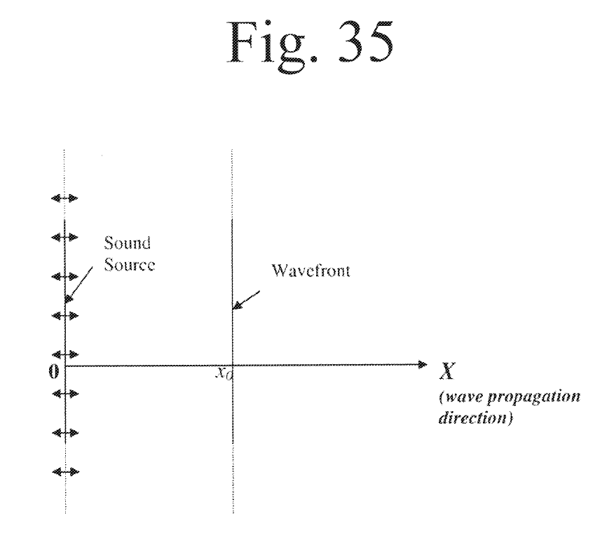

### DETAILED DESCRIPTION OF THE INVENTION

### Transducer Design

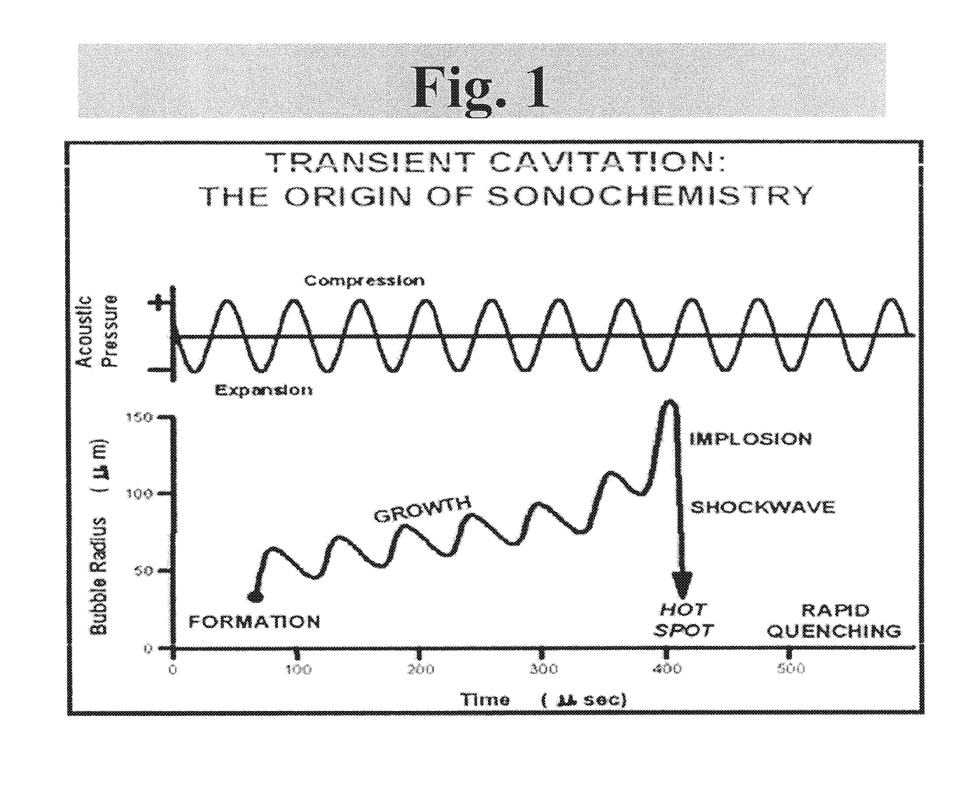

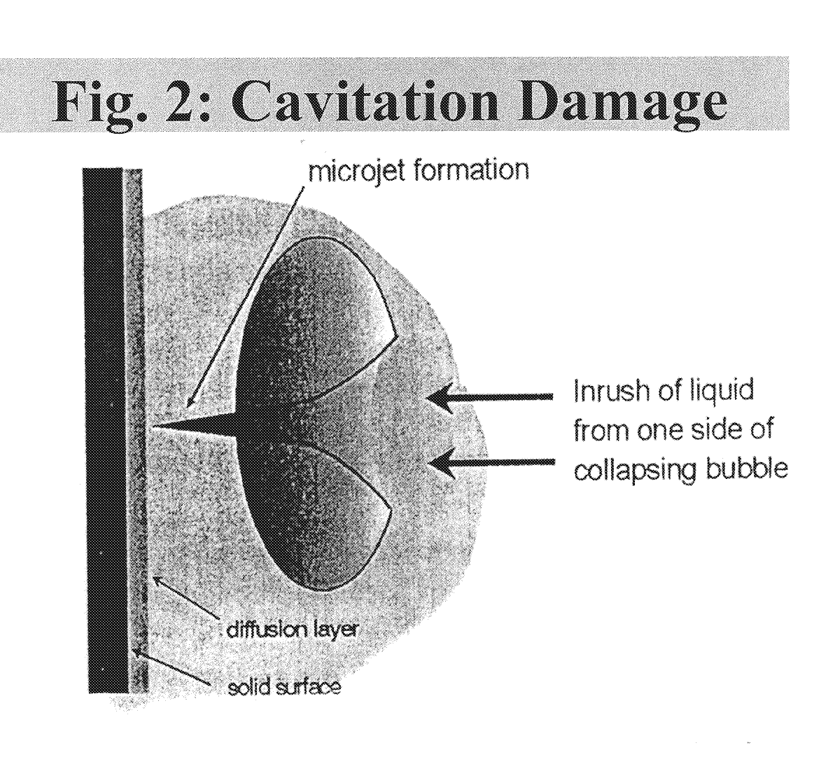

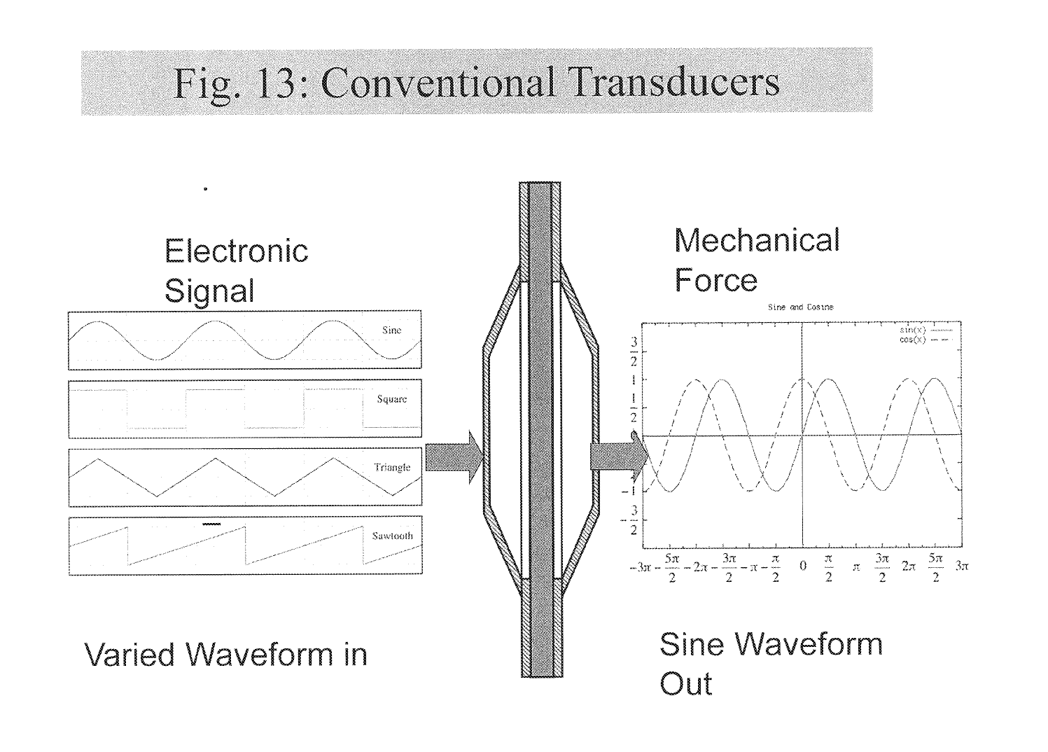

In FIG. 13 the function of a conventional piezoelectric transducer, which is designed traditionally employing a piezoelectric crystal which converts an electronic signal into mechanical vibratory energy. No matter the electronic signal waveform delivered to the transducer, a sinusoidal ultrasonic waveform mechanical force is generated, creating the cavitation effect depicted in FIGS. 1 and 2.

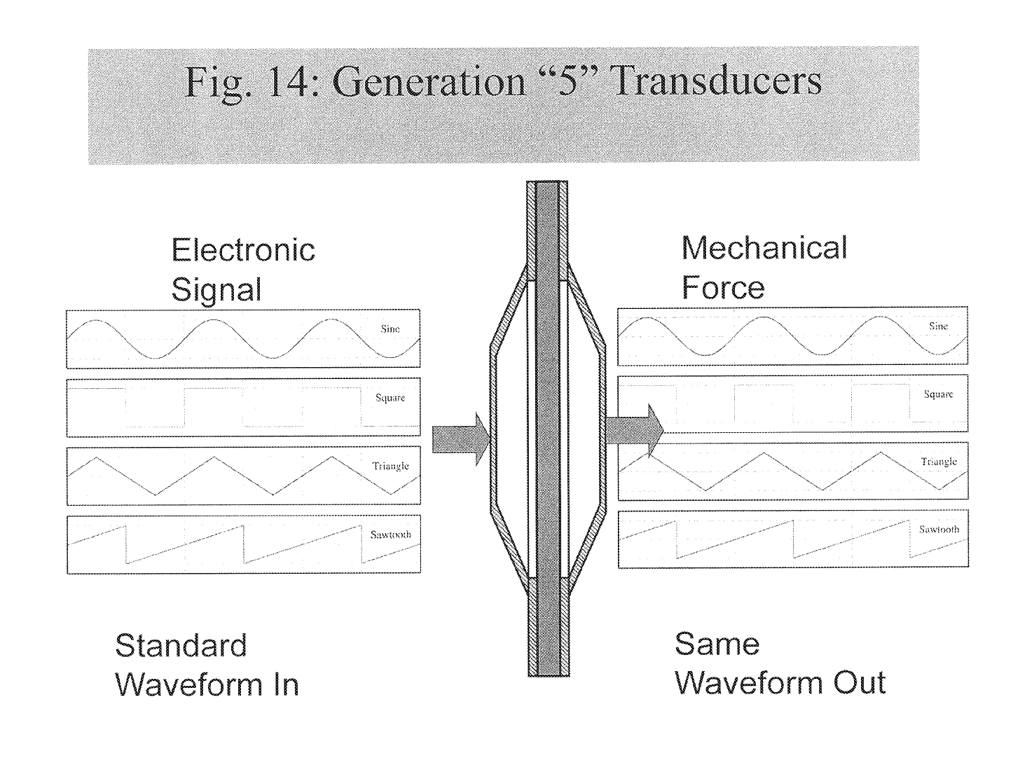

FIG. 14 illustrates the function of a modified transducer, wherein the electronic signal delivered to the transducer is repeated purely as mechanical force upon the output of the transducer. A sinusoidal electronic transmission is delivered as an ultrasonic sinusoidal waveform mechanical force output. Similarly a sawtooth, triangular or square waveform electronic transmission is delivered as an ultrasonic sawtooth, triangular or square waveform mechanical force output, respectively. This type of transducer eliminates or minimizes the formation of micro-bubbles and cavitation and resultant heat, which could damage a drug or the skin.

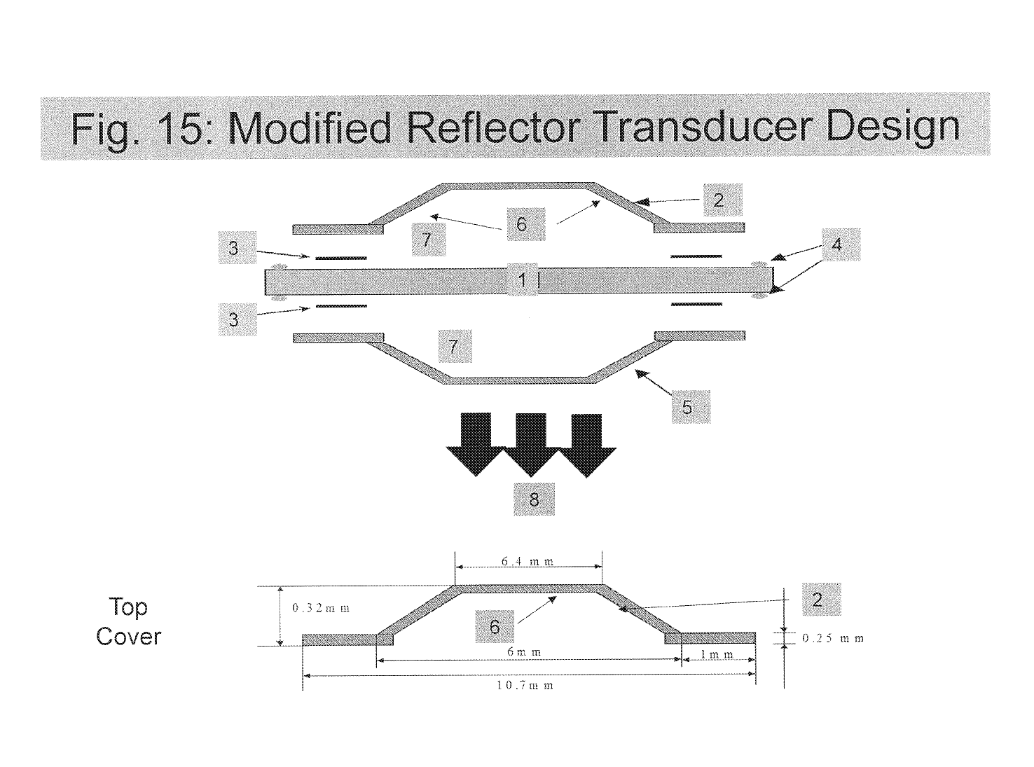

FIG. 15 is a schematic design of a modified transducer which will create the alternating ultrasonic transmission as depicted in FIG. 14, wherein the mechanical sonic waveform follows the electronic waveform delivered to the piezoelectric crystal.

In particular the transducer consists of a piezoelectric crystal or a magnetorestrive crystal (1) in FIG. 15, which is sandwiched between two cover layers which control the vibratory direction of mechanical force emitted from the crystal (1). At the bottom of the transducer a sonic film layer (5) allows the sonic signal to pass through it undeterred. On top of the crystal (1) a reflective, non-sonic permeable material (2) reflects mechanical force back through an air gap (7) which is between both film coverings (2) and (5). The covers (2) and (5) encapsulate the crystal (1) and are connected by a flexible rubber connector, such as a sponge foam connector (3) which is placed between the top cover (5) and the bottom cover (2). A rubber stop or gasket (4) is placed on both sides and seals the entire transducer into place.

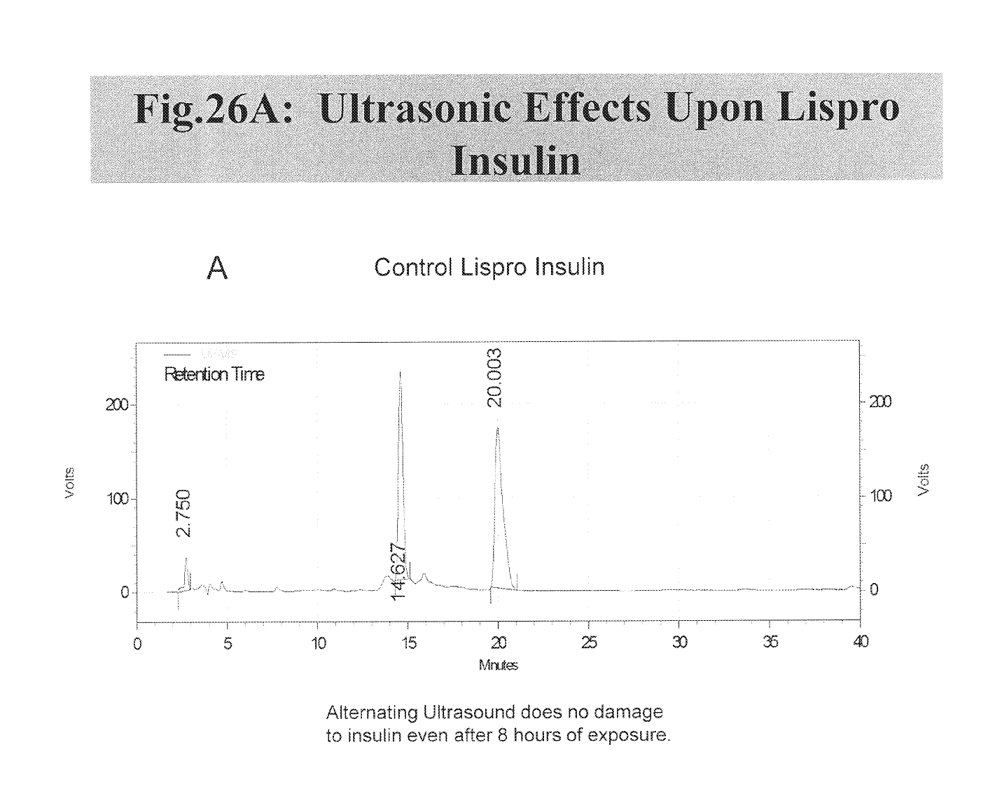

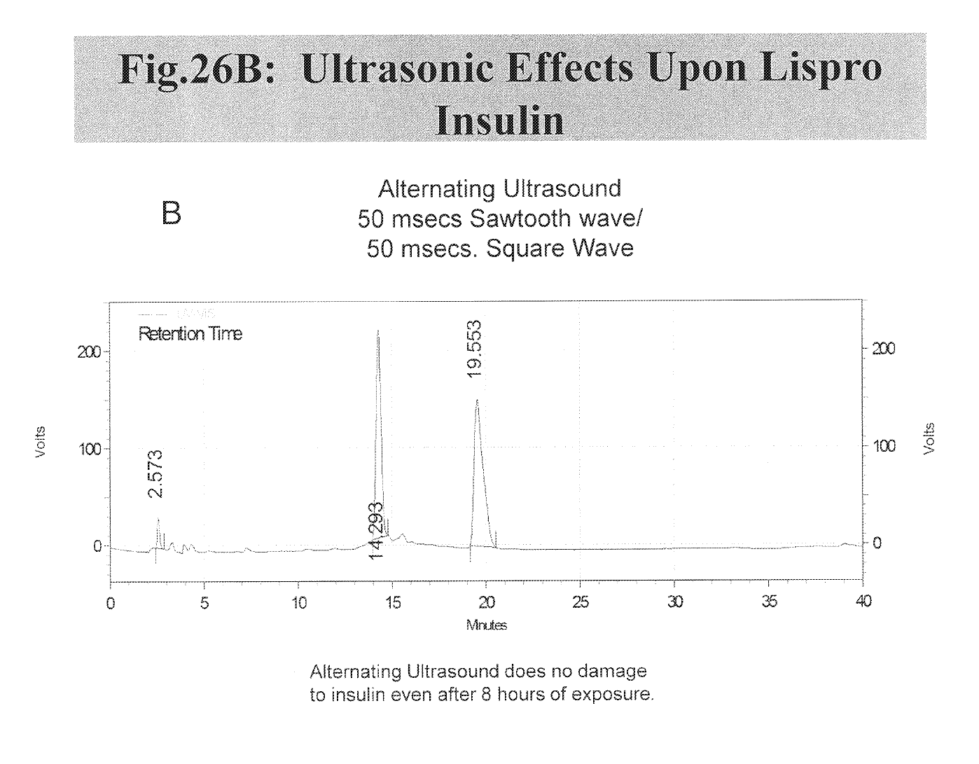

Electrical energy delivered to the crystal (1) causes it to vibrate mechanically and develop ultrasonic force. That mechanical force travels through the air gap to the top of the transducer where it is reflected back downwards by the material at the top cover (2), back through the top air gap (7) to the bottom of the transducer where the mechanical energy passes through the bottom cover (5) and exits the transducer as ultrasonic force (8). As the crystal (1) vibrates it flexes the rubber stop (4) and the sponge foam connector (3) allowing the entire cover, both (2) on top and the bottom cover (5) to vibrate harmonically with the vibration of the crystal (1). The result is an intense ultrasonic transmission, which delivers a waveform shape commensurate with the electrical waveform delivered to the transducer as seen in FIG. 26B.

The top cover (2) is designed to reflect ultrasonic energy back downward through the bottom of the transducer. Conventional transducers deliver ultrasound in all directions, lowering their overall intensity. The preferred material for the top cover (2) is a titanium foil. On the interior of the foil an insulating coating of epoxy resin is placed to enhance the ability of the titanium foil to remain rigid and non-harmonically reactive to the ultrasound emanated from the crystal (1). By re-focusing the sonic energy downward, the top cover enhances the intensity of the sonic transmission and avoids waste of the energy. The use of sponge foam connector (3), which is placed between the top cover (2) and the bottom cover (5) coupled with the rubber stop (4) allows the transducer to flex, much like a speaker, with the ultrasonic transmission (8), resulting again in a stronger more intense transmission. The slight air gap (7) between the covers (2) and (5) and the crystal (1) avoids complete rigidity for the transducer and enhances its flexing capacity. The result is a high intensity transducer which will require less energy to power it and which performs the function of delivering the mechanic ultrasonic waveform, matching the electrical waveform delivered to the transducer.

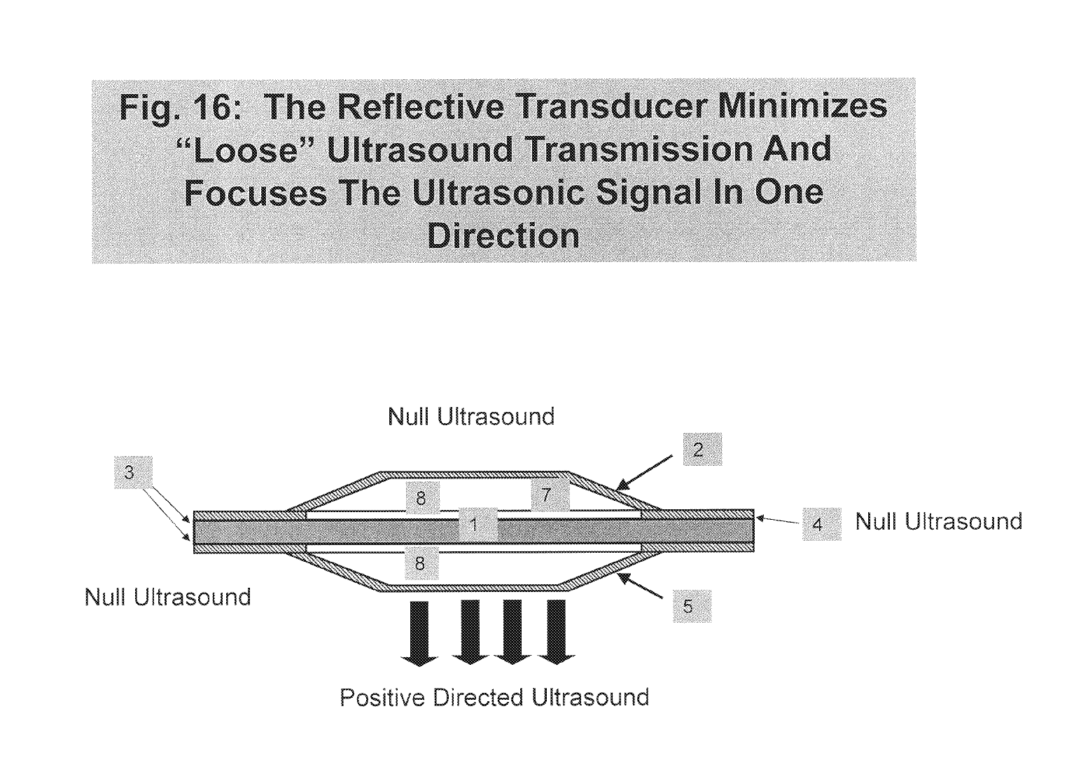

In FIG. 16 it can be seen that the transducer delivers null or very little ultrasound out the top or sides of the transducer while most of the energy is directed downward from the bottom of the transducer, forming a directional ultrasonic transmission.

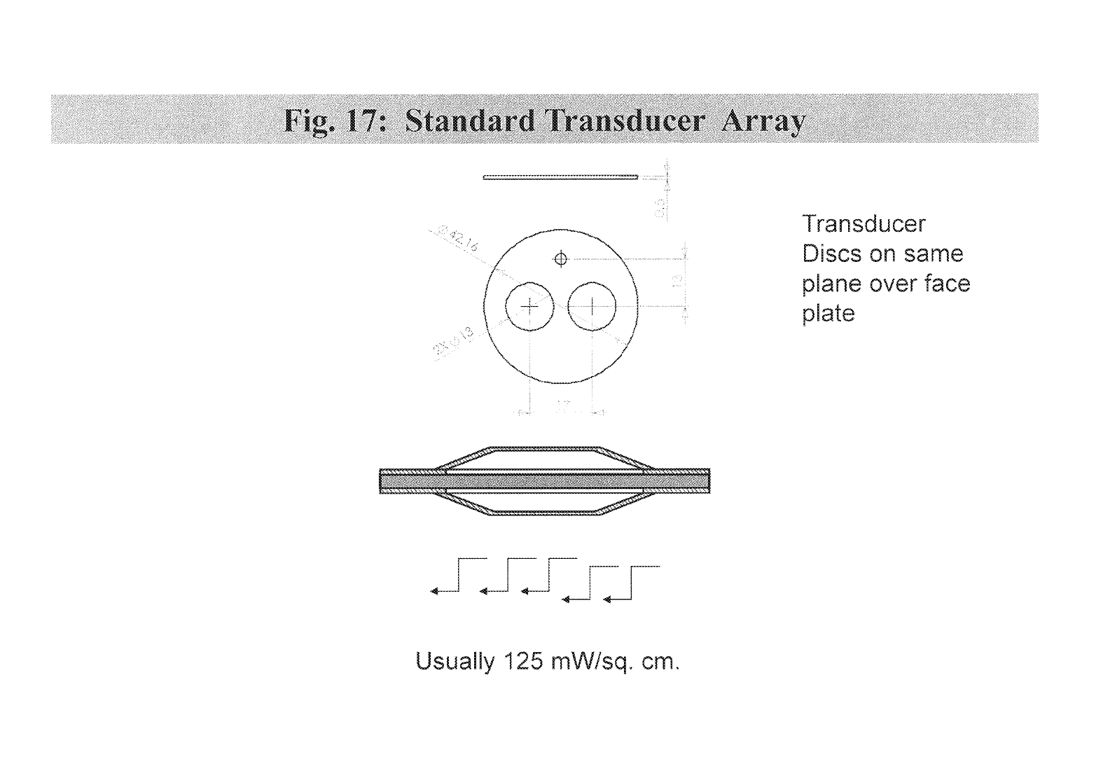

In FIG. 17 the transducer discs are generally constructed on a single plane. FIG. 17 depicts two transducer discs affixed onto a stainless steel face plate all on one level making what is termed as a Standard Transducer Array.

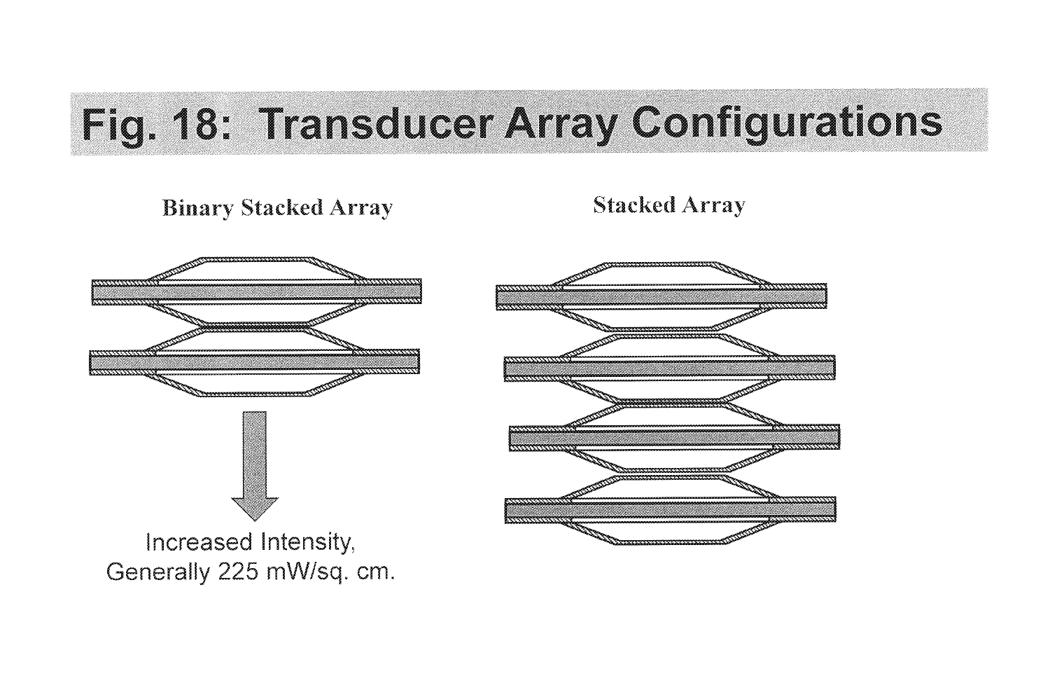

FIG. 18 illustrates a stacked array which may consist of two transducers (a binary stacked array) or a stacked array, which is several transducers placed on top of one another. The stacked array can increase the intensity of the ultrasonic transmission. The use of stacked transducers, essentially transducers fitted on top of each other, increases ultrasonic intensities while maintaining a given frequency level. Used in this invention, the stacked transducer construction is intended to increase intensity while improving the power utilization of the transducer system.

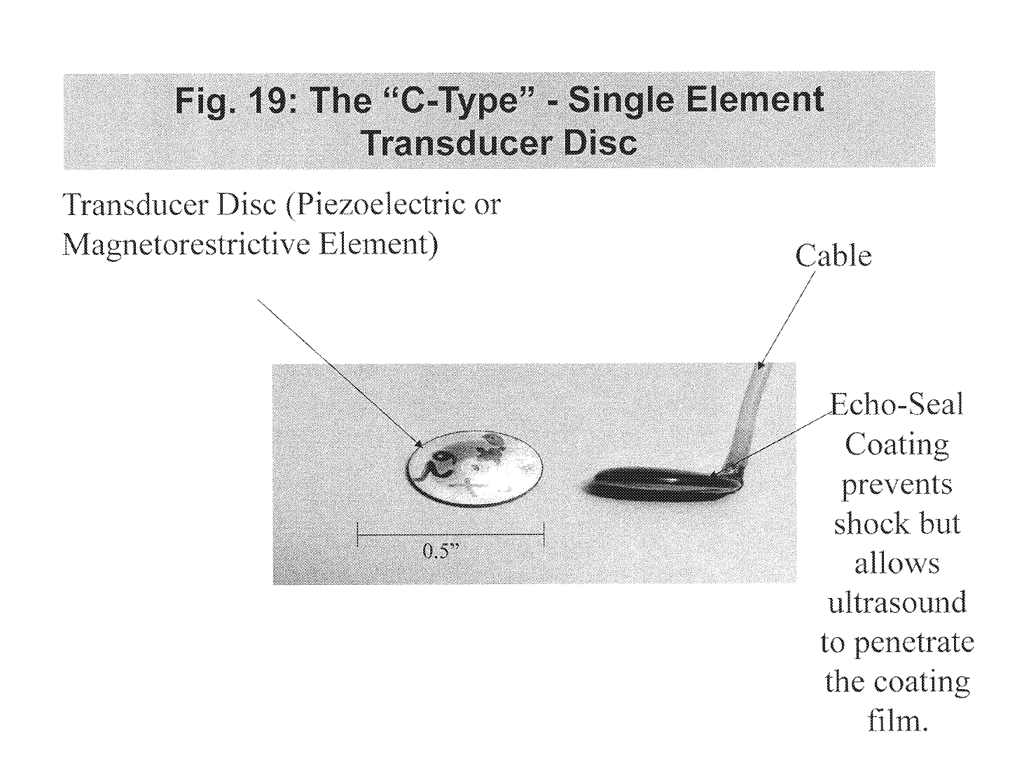

FIG. 19 illustrates that the “C” type transducer disc enables a compact and minute size for the transducer element of the invention. The sizing of the transducers was obtained at just 0.5 inch in diameter. The small size transducer was used in the invention to enable the transducers to fit within the dimensions of transdermal patches for drug delivery applications but have many other uses, and can have other sizes. In addition, the small size enabled a lower weight potential for the transducers, again aiding in the portability of the invention.

The transducer disc is a “C” type construction attached to a power cable. The transducer disc is coated in a polymer housing, ideally composed of URALITE™ urethane resin and referred to an Echo-Seal resin, which is used to avoid short circuits between the two metallic caps (FIG. 15) and provides acoustic coupling for the sonic transmission.

### Design of Transducer Element or Disc

FIG. 15 illustrates the design of ultrasonic transducer, which is the preferred embodiment of the transducer element of this invention. From FIG. 15 it can be seen that a transducer 40 is based upon a piezoelectric disc (1), such as available as PZT4 (Piezokinetics Corp. Bellefonte, Pa.), connected between two metal caps (2) and (5) composed of titanium foil preferably, without limitation. A hollow air space (7) is between the piezoelectric disc (1) and the end caps (2) and (5). The end caps (2) and (5) are connected to the piezoelectric disc (1) by a non-electrically conductive adhesive (3) to form a bonded layered construction to the transducer (4). A polymer coating (6) is placed on the inside of the top and bottom end caps (2) and (5) and helps minimize harmonic reaction of the end caps to the ultrasound generated from the disc (1). End cap (2), with the help of the internal coating (6), acts a reflector directing the ultrasound in one direction, shown by the arrows (8), at the bottom side of the transducer.

The transducer offers a thin, compact structure more suitable for a portable ultrasonic drug delivery apparatus. Additionally, this transducer offers greater efficiency for the conversion of electric power to acoustically radiated power. This design of a transducer was also chosen because of its potential to be battery powered and its small, lightweight features.

FIG. 16 shows that the design illustrated in FIG. 15 has sonic energy emanating in one direction from the transducer and not at the top or at the sides.

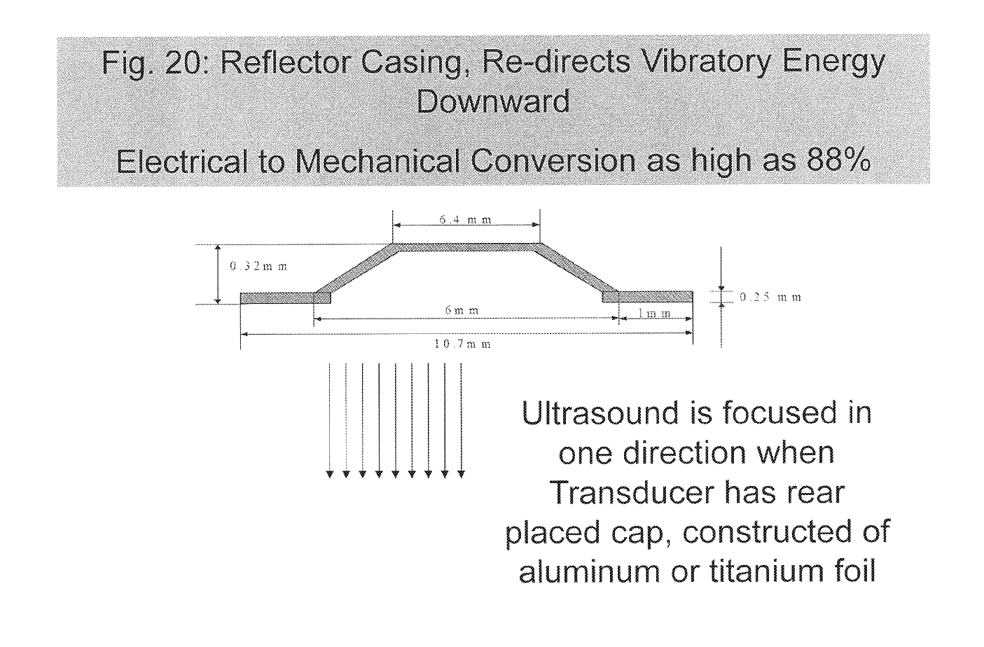

FIG. 20 shows that the design illustrated in FIG. 15, through the use of the caps achieves a high efficiency of electrical to mechanical conversion of sonic energy, as high as 88% when traditional cavitation based sonic transducers generally have efficiency as low as 18%. The reflector end cap directs the vibration in one direction.

### Fabrication of the “C Type” Transducer-Standard Construction as Illustrated in FIG. 15

### Part List and Step By Step Manufacturing

### PARTS LIST

1. Piezoelectric ceramic Material:
PZT4 disc 0.5-inch diameter, 1-mm thickness (PKI402)
SD 0.500-0.000-0.040-402
Actual supplier: Piezo Kinetics Inc.
Mill Road and Pine St.
PO Box 756
Bellefontte Pa. 16823
2. Titanium caps
Material: Alfa Aesar, Titanium Foil, 0.25 mm thick, metal basis 5%, Item #10385
Actual supplier: Alfa Aesar, A Johnson Matthey Company 30 Bond Street Ward Hill, Mass. 01835-8099, USA
3. Bonding layer
Material: Eccobond 45LV+catalyst 15LV
Actual supplier: Emerson & Cuming
46 Manning Road
Billerica, Mass. 01821
4. Low temperature soldering
Material: Indalloy Solder #1E, 0.30″ dia x 3 ft long
Actual supplier: The indium corporation of America 1676 Lincoln AVE UTICA N.Y. 13502
5. Wires
Material: Stranded wire, Gauge/AWG: 30
Catalog number (Digikey): A3047B-100-ND
Note: Maximum Temperature: 80 C
Conductor Strand: 7/38
Voltage Range: 300V
Number of Conductors: 1
Actual supplier: Alpha Wire Corporation
6. Housing polymer
Material: Uralite FH 3550 part AB
Actual supplier: HB Fuller Company
7. Ethyl Alcohol
Note: 200 proof (at least)
8. Sand paper

Manufacturing Procedure: Step-by-Step

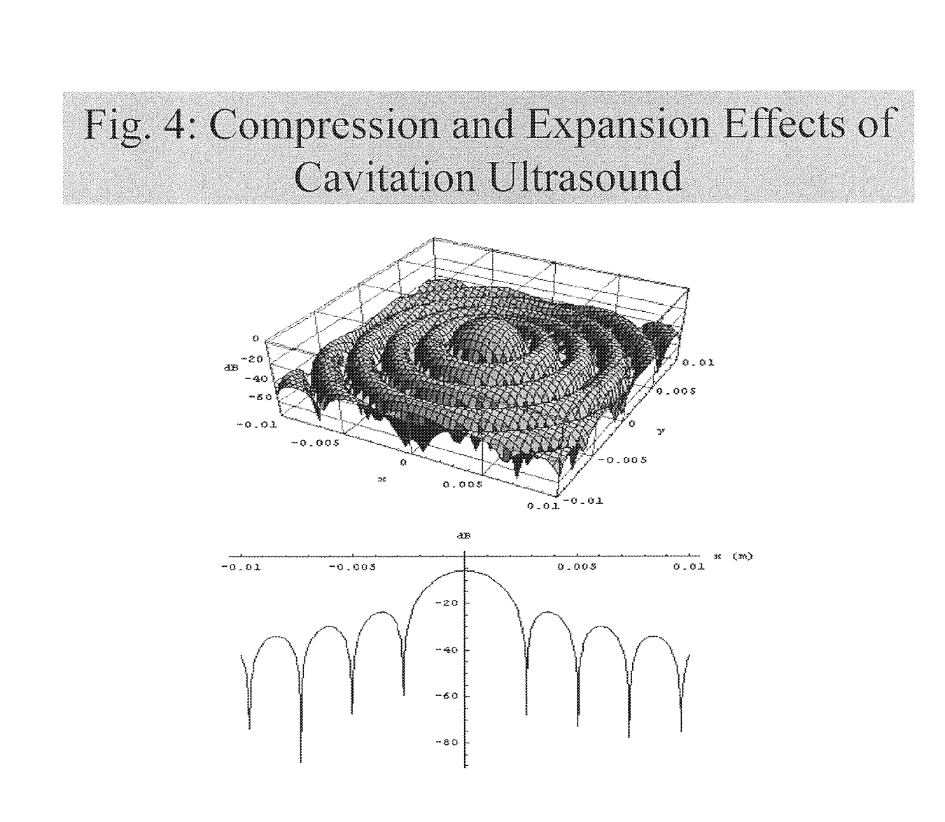

Reference is made to FIG. 4B:

1. Dye cut titanium foils into several disks. Materials: Titanium foil (2), circular saw 10.7 mm diameter.

2. Sand rough edges. One side of the disks results with edges. Those edges should be removed with sand (fine scale) paper. Materials: Sand paper (8)

3. Alcohol bath to remove dust generated by sanding the disks. Materials: alcohol (7)

4. Put disk into a high pressure (12000 torr) shaping tool (polished side up). For this step should be designed a custom-made punch dye in order to shape the disks into the dimensions reported in FIG. 2.

5. Sand rough edges again. Materials: sand paper (8)

6. Immerse in alcohol to remove dust. Materials: alcohol (7)

7. Wipe to remove alcohol and dust from disk

8. Measure thickness of cap with special measuring pen

9. Identify matching caps (by thickness). This step should be accurate because slight differences between the two caps generate spurious resonance into the C Type.

10. Clean piezo disk ceramic with alcohol. Materials: piezodisks (1) and alcohol (7).

11. Screen printing on both sides with epoxy bond. Materials: bonding epoxy (3) and a tool for screen-printing (like T-shirt screen-printing). We should generate a ring of epoxy to glue the caps with the disks. This ring should be accurate and regular in order to avoid spurious resonance.

12. Place C Types on ceramic disk

13. Place into a press. This press should just keep the C Type made in place. It could be a custom-made tool where several C Types are kept in place.

14. Place press into oven for at least 4 hours, 70 Celsius

15. Solder at maximum 270° C. at 4 points per piece. Materials: wires (5) and low temperature solder (4).

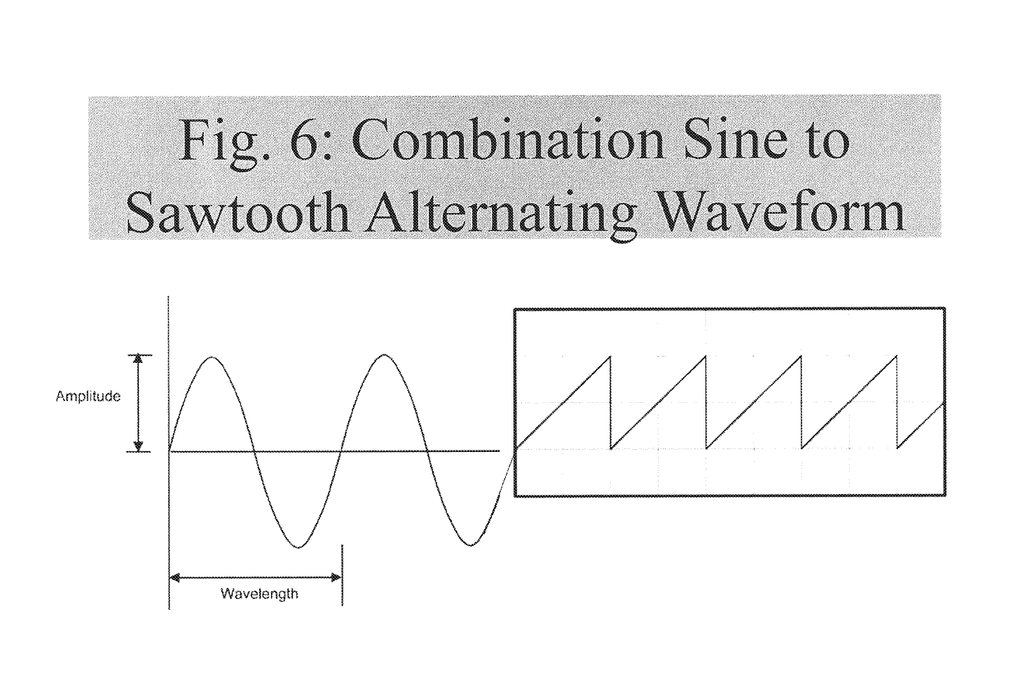

The transducer produced by the above procedure is a standard construction. To form a stacked array construction transducer two or more transducers are placed directly atop one another as shown in FIG. 4C and fitted together. To form an array the transducers are generally connected in parallel electrically within the polymer or epoxy bonding material as shown in FIG. 6, in either single element form or in a stacked construction format.

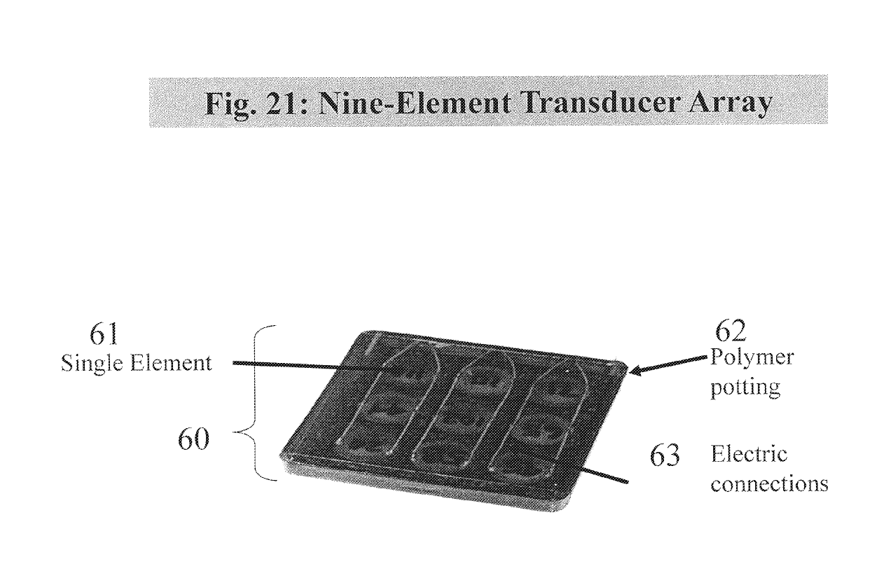

FIG. 21 illustrates the original design of the transducer array with nine transducer discs encased in an epoxy block.

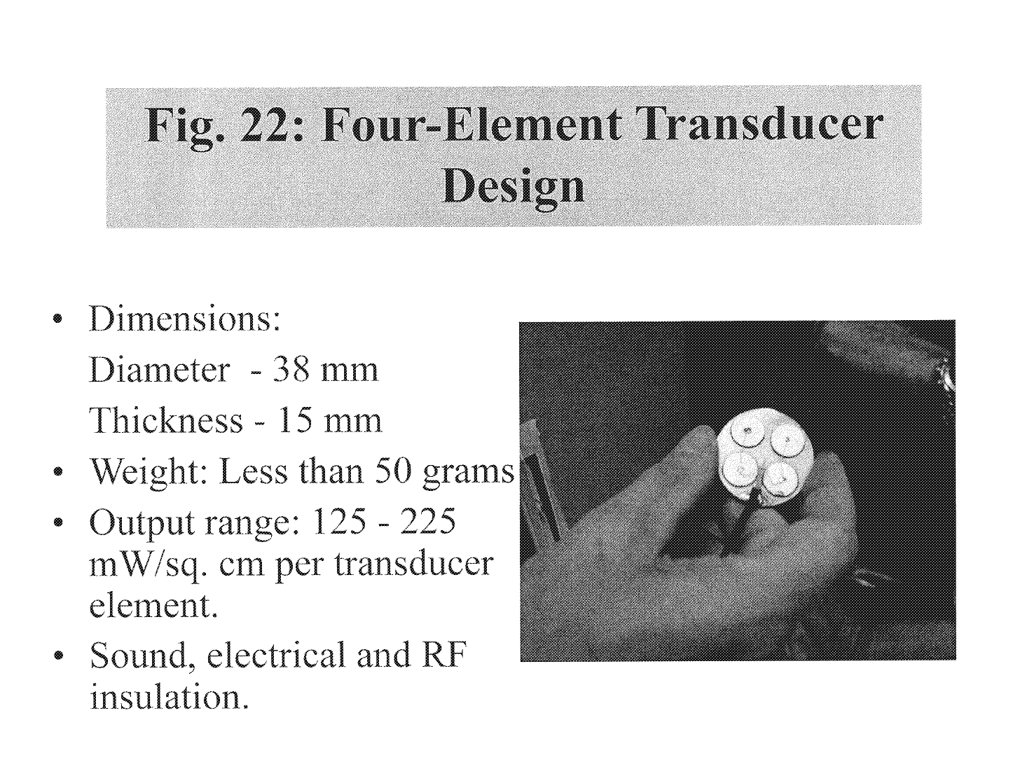

FIG. 22 shows the final design which is four transducer discs attached to a stainless steel face plate. In the design shown in FIG. 21, there are nine separate ultrasonic transmission form the block, over each transducer discs. In FIG. 22 the four transducer discs develop a harmonic between their individual transmissions and cause the face plate to deliver a uniform, single, larger transmission over a larger transmission area.

### Transducer Block

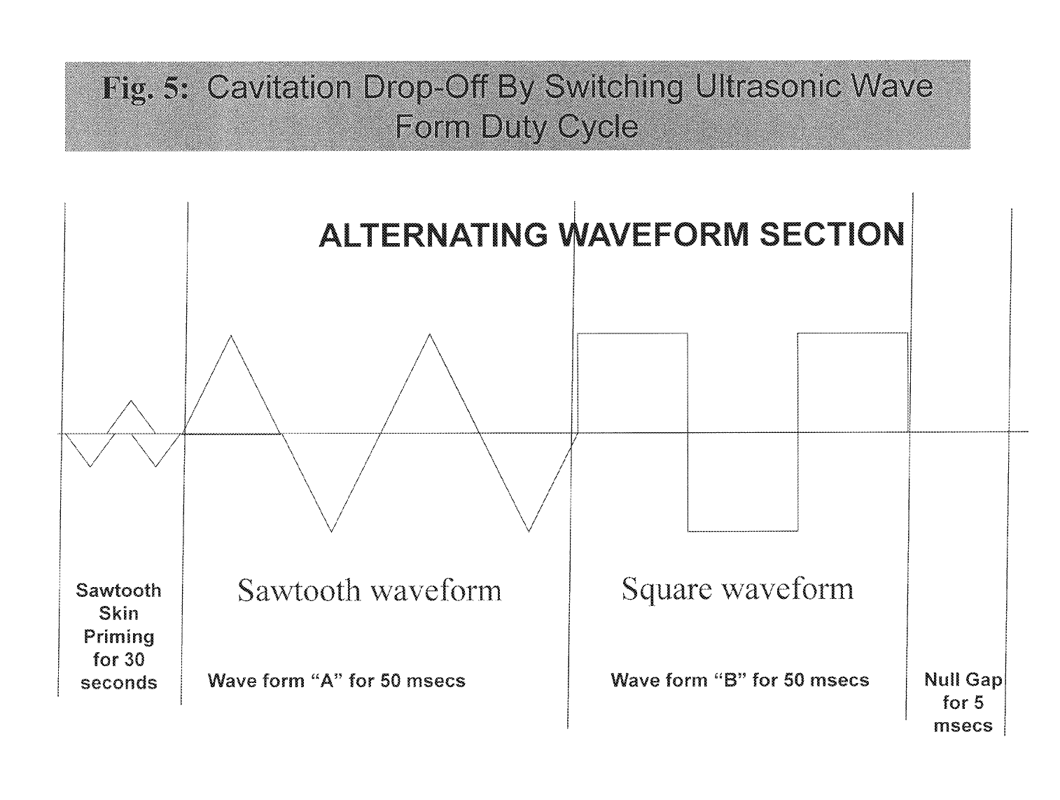

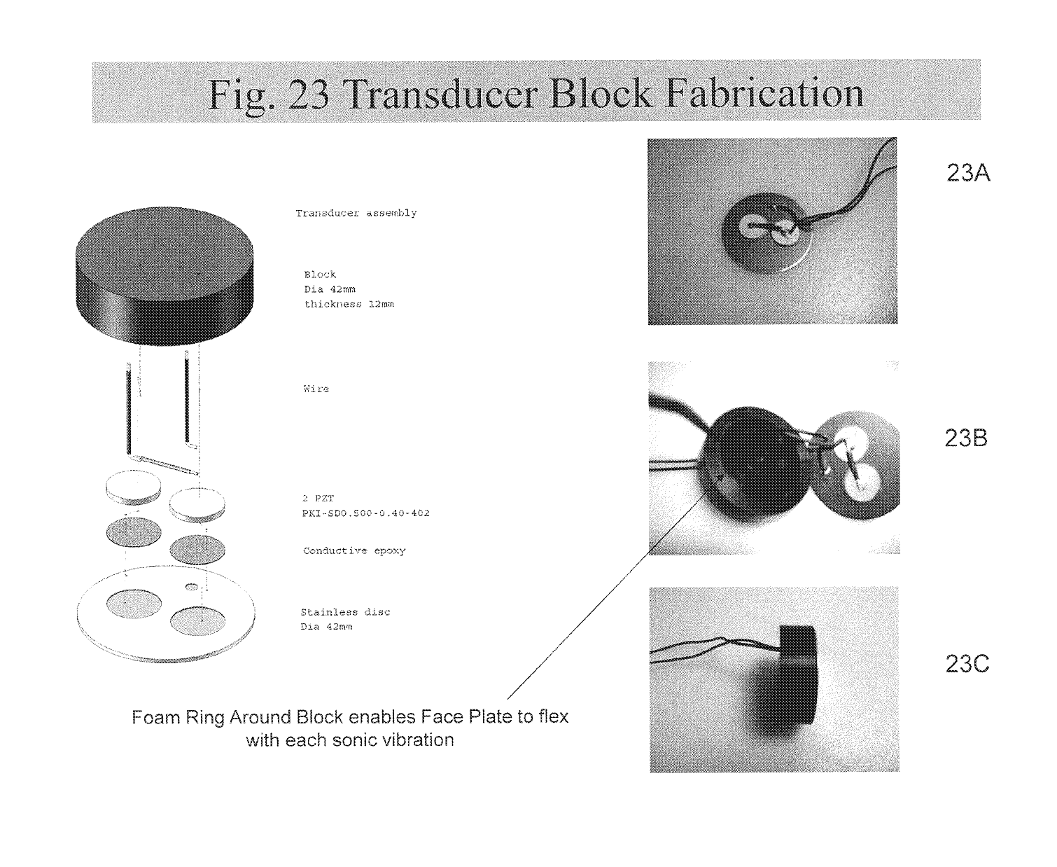

FIG. 23 is a schematic design of a modified transducer which will create the alternating ultrasonic transmission as depicted in FIG. 5. FIG. 17 is a an array of two transducer discs affixed to a stainless steel face plate, and covered by a block material which assists in the direction of the ultrasound transmission through the face plate and toward the target. FIGS. 23 A, B and C show the assembly steps for this block transducer array.

### Experiment—1

Temperature Comparison Between a Sinusoidal Vs. The Alternating Ultrasonic Transmission in Tap Water

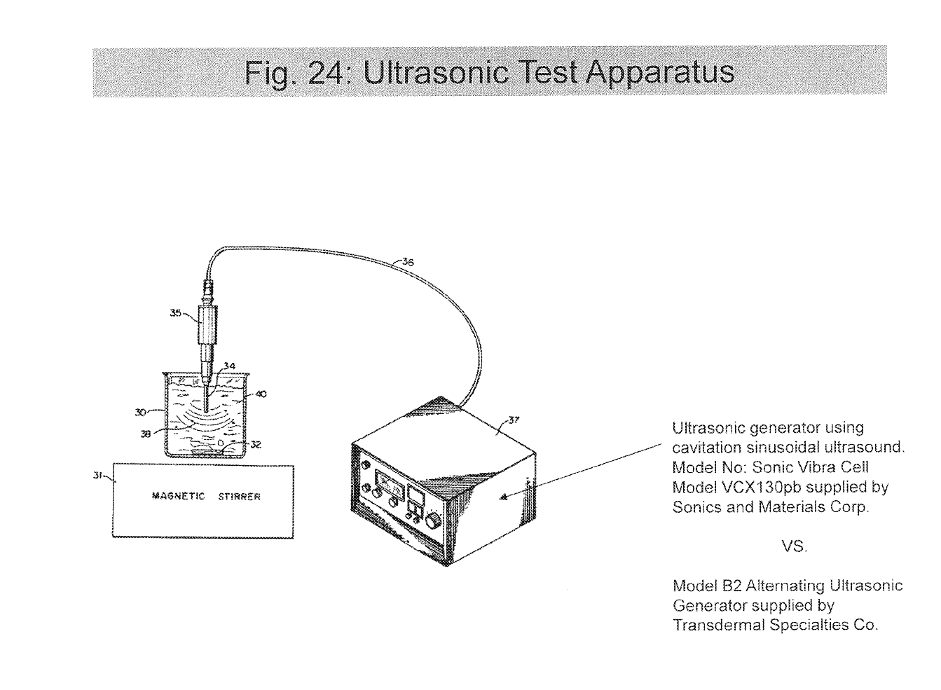

Refer to the configuration depicted in FIG. 24. A glass beaker (30), containing 1,000 mls of tap water (40) was placed atop a magnetic stirrer (31). Inside the beaker a magnetic stir bar (32) was made to slowly rotate within the water.

An ultrasonic probe (35) was placed into the water using an ultrasonic single transducer tip (34). The tip can be a sinusoidal ultrasonic tip or one practicing this invention, which generates an ultrasonic alternating waveform transmission (38). The ultrasonic generator (37) powered the ultrasonic probe (35) through a cable (36).

Using a Sonic Vibra Cell Model No VCX 130 pb, manufactured by Sonics and Materials Inc., Newtown, Conn., as an ultrasonic generator (37), which is a sinusoidal ultrasonic generator and probe, temperature comparison tests were made vs. a B2 Alternating Ultrasound generator made according to the present invention by Transdermal Specialties, Inc., Broomall, Pa. The alternating system employed the ultrasonic 4-element array depicted in FIG. 22, while the conventional probe only had one element at the tip.

After 5 minutes of ultrasound application to 1,000 ml of tap water, the Vibracell system exhibited a 5.5° C. rise, evidence of intense cavitation.

After 5 minutes of ultrasound application to 1,000 ml of tap water, the B2 Alternating Ultrasound generator produced a −0.9° C. change in temperature, a drop of −0.9 degrees. Essentially there was no change in the temperature of the water within the beaker, the slight downward temperature resulting from the water sitting out. If there had been any cavitation generated from the alternating system the temperature would have risen.

### Experiment 2

### Temperature Comparison Between a Sinusoidal Transducer Vs. Fluid Mobility Caused by the Alternating Ultrasonic Transducer

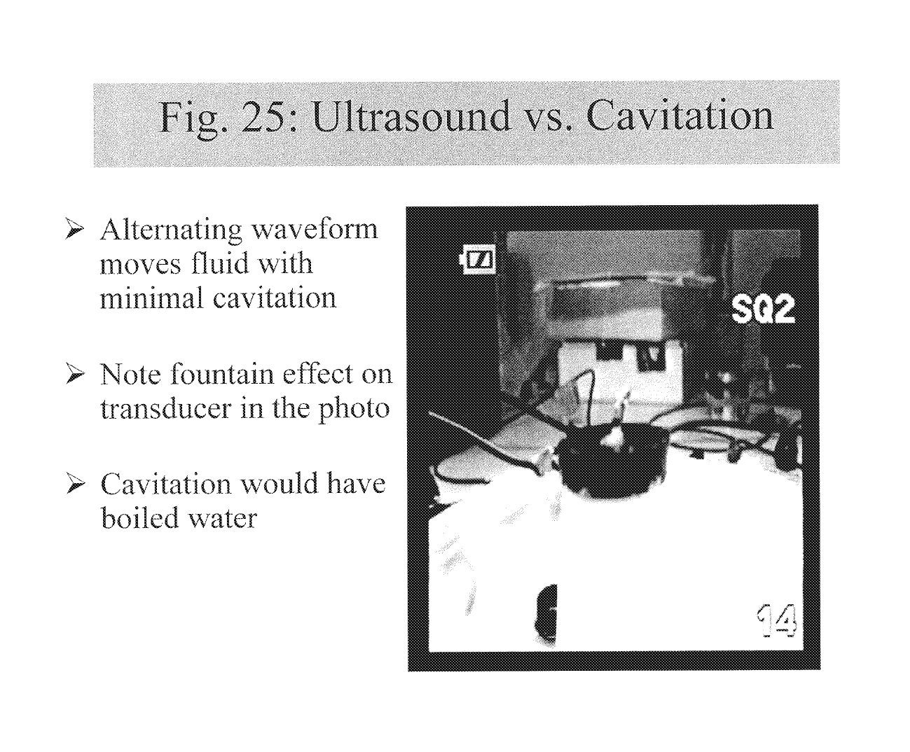

Referring to FIG. 25, this experiment placed one gram of tap water on the surface of a transducer and observed the effects.

In a first run, a Sonic Vibra Cell Model No VCX 130 pb, manufactured by

Sonics and Materials Inc., Newtown, Conn., the conventional probe only had one element at the tip, which is a sinusoidal ultrasonic generator and probe temperature comparison tests, was used, upside down, to determine what the visual effect would be on one gram of water. The observation indicated very fast conversion from a liquid state to steam, an indication of intense cavitation.

Repeating the experiment using a B2 Alternating Ultrasound generator according to the present invention made by Transdermal Specialties, Inc., Broomall, Pa., the alternating system employing the Ultrasonic 4-element array depicted in FIG. 22, resulted in a fountain that actually pushed the water from the surface of the transducer. No appreciable heat was detected and no steaming was observed.

These tests showed that the alternating ultrasonic transmission again demonstrated no cavitation force, but also demonstrated a vibratory force which moved the liquid vertically from the transducer array.

### Experiment 3

### A Series of HPLC Spectrographs were Taken of Lispro Insulin Subjected to Either Sinusoidal Ultrasound or to the Alternating Ultrasonic Waveform Transmission

In graph of FIG. 26A, it can be seen that 1 gram of Lispro insulin has an HPLC spectra shown as control, in that insulin is not subjected to ultrasound.

In the FIG. 26B graph, 1 gram of Lispro insulin was subjected to the alternating ultrasound transmission, over 8 hours of continuous exposure, at 50 msecs sawtooth followed by 50 msecs square wave. This experiment produced an HPLC spectra identical to the control, indicating no degradation of the insulin.

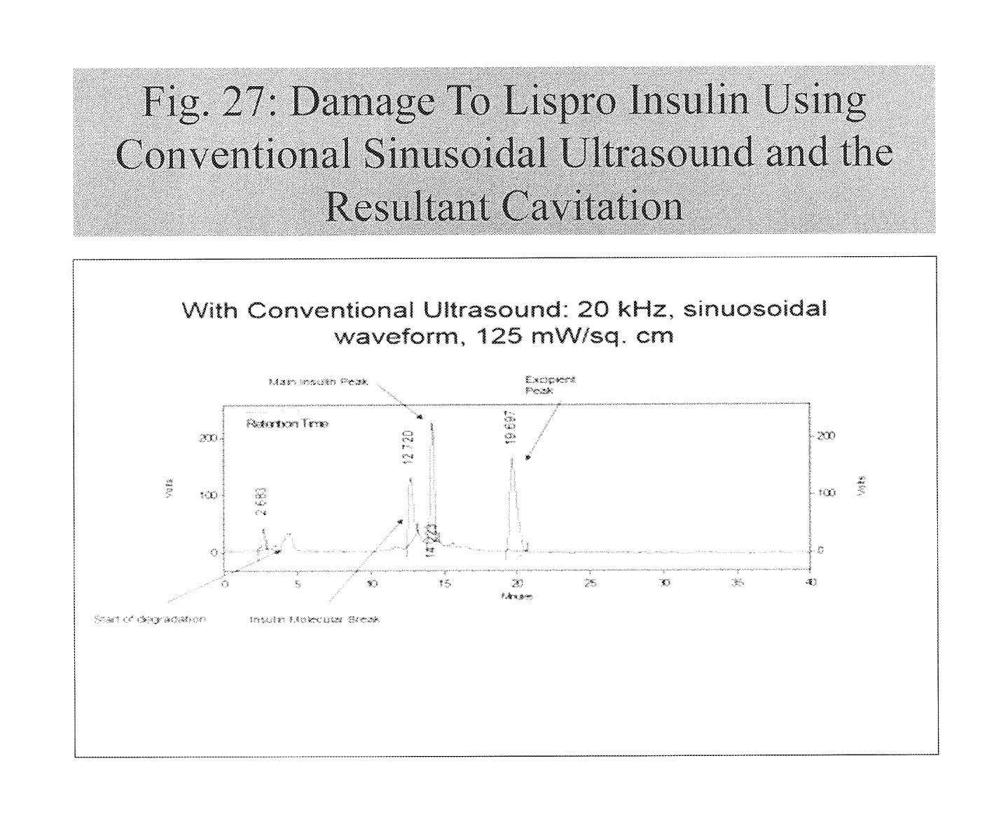

FIG. 27 shows damage to the insulin caused by a sinusoidal ultrasound transmission as effected to 1 gram of Lispro insulin, using a Sonic Vibra Cell Model No VCX 130 pb, described in the previous experiments, using a conventional sonic tip, which is a sinusoidal ultrasonic generator. The exposure was just 1 minute. In this case the insulin HPLC spectra showed severe degradation of the drug. This is due to cavitation. The temperature of the drug rose by 4.3° C. over a 1 minute exposure.

### Experiment 4

### Use of Alternating Ultrasonic Transmission to Effect Pore Dilation in the Skin to all the Delivery of Large Molecule Substances

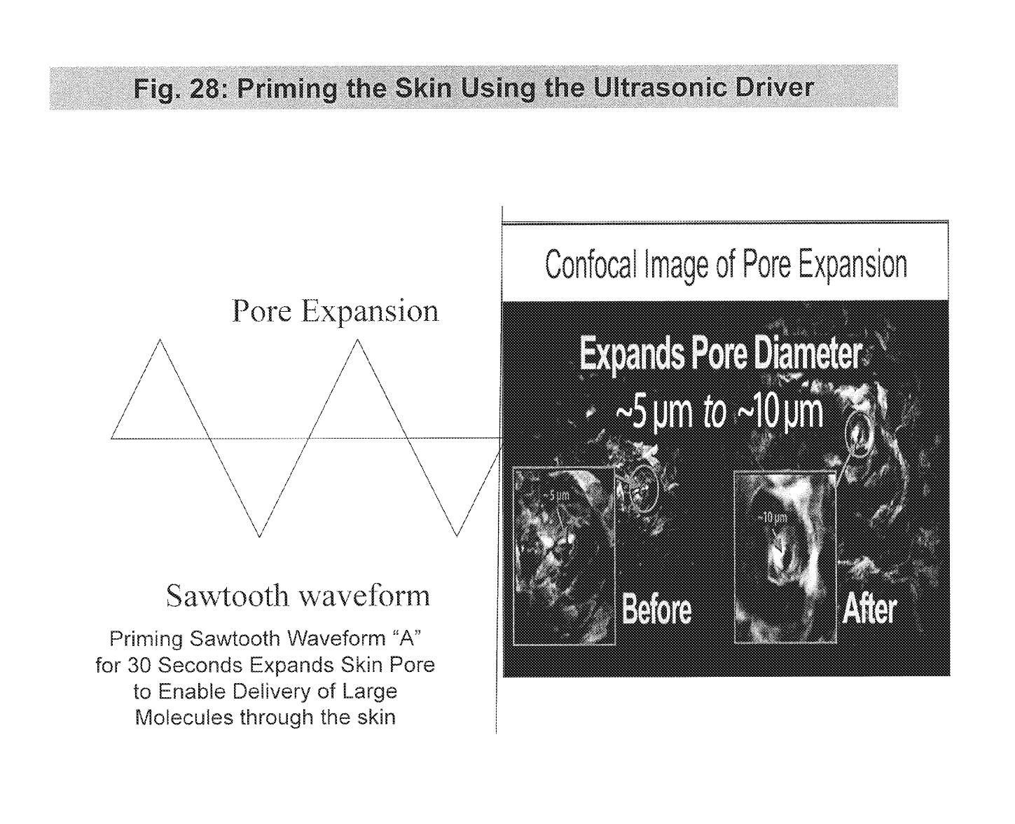

FIG. 28 shows pore dilation of human skin as effected by the use of the alternating ultrasonic waveform. It is believed the sawtooth component exerts a horizontal force upon the skin which acts to dilate the skin pores and expand the opening from 5 to 10 microns, using cadaver facial skin.

### Experiment 5

### Beam Analysis and Comparison Between Cavitation Ultrasound Vs. The Alternating Ultrasound Transmission

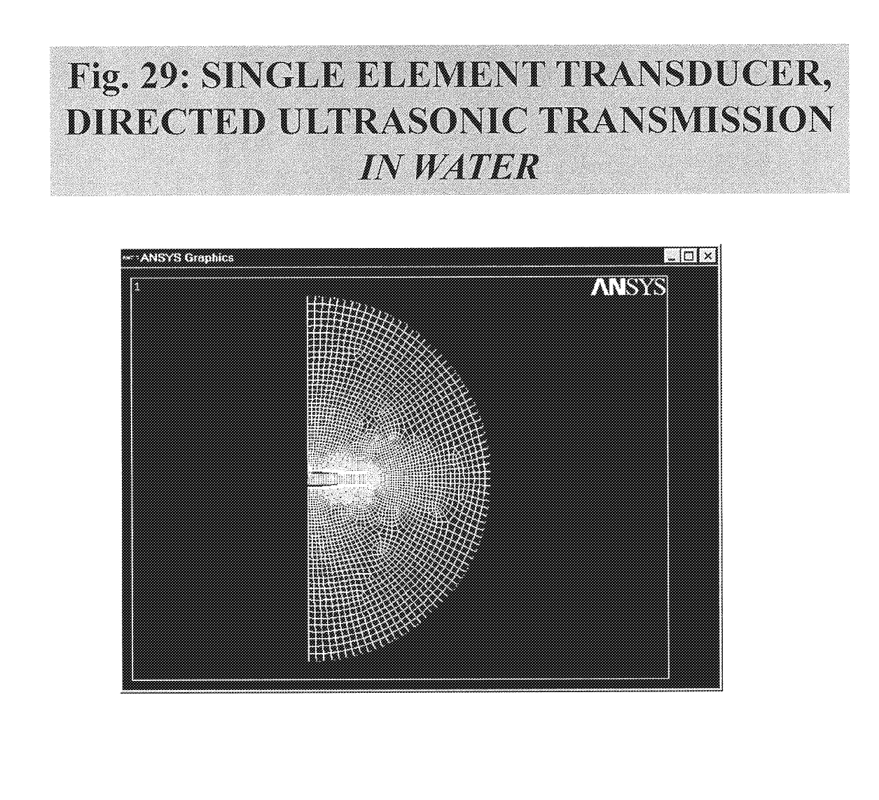

FIG. 29 illustrates the beam transmission of a single element transducer configured according to the four-element transducer design according to the present invention depicted in FIG. 22, which propagates a 50 msec sawtooth followed by a 50 msec squarewave alternating transmission according to the design depicted in FIG. 5.

As depicted in FIGS. 15 and 16, the ultrasonic transmission in colored water (FIG. 29) shows the transmission was emanated in one direction from the transducer.

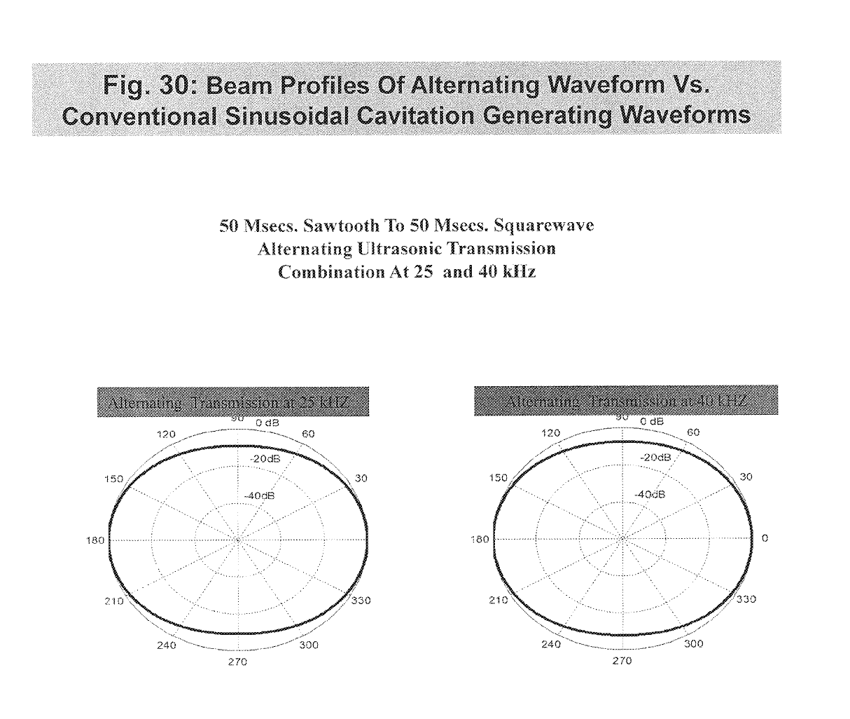

Looking at the beam profile of the ultrasonic transmission upon contact with paper, the alternating transmission at 25 kHz and 40 kHz frequency shown in FIG. 30 shows a nearly uniform spherical transmission pattern in two separate experiment runs.

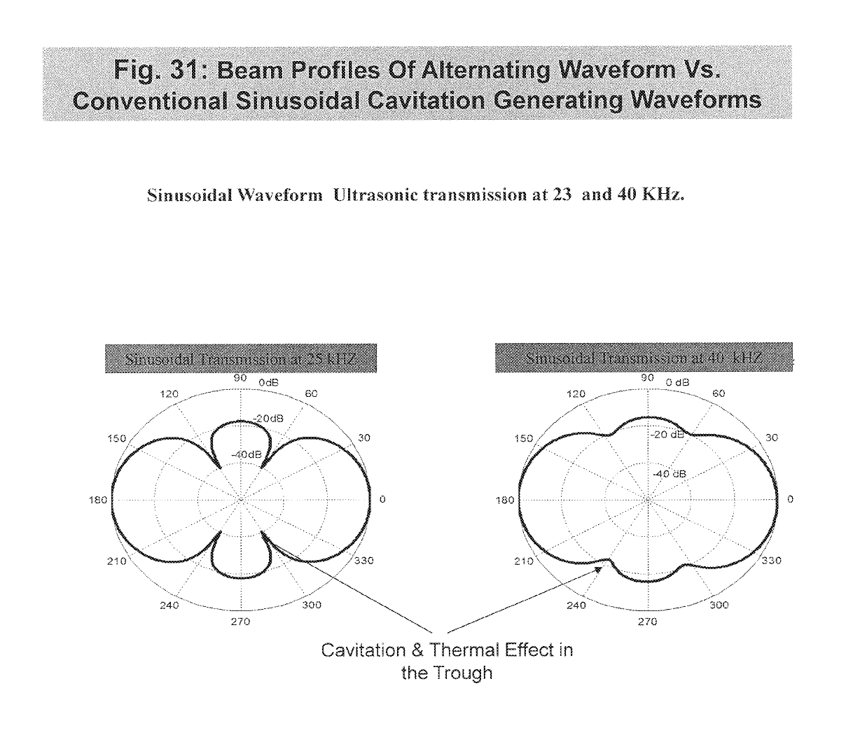

FIG. 31 illustrates a beam profile at 24 and 40 kHz using a sinusoidal ultrasonic transmission. The beam profile is odd shaped and intense heating effects are apparent at the intersection point on the patterns. The cavitation was more intense at 25 kHz and less at 40 kHz.

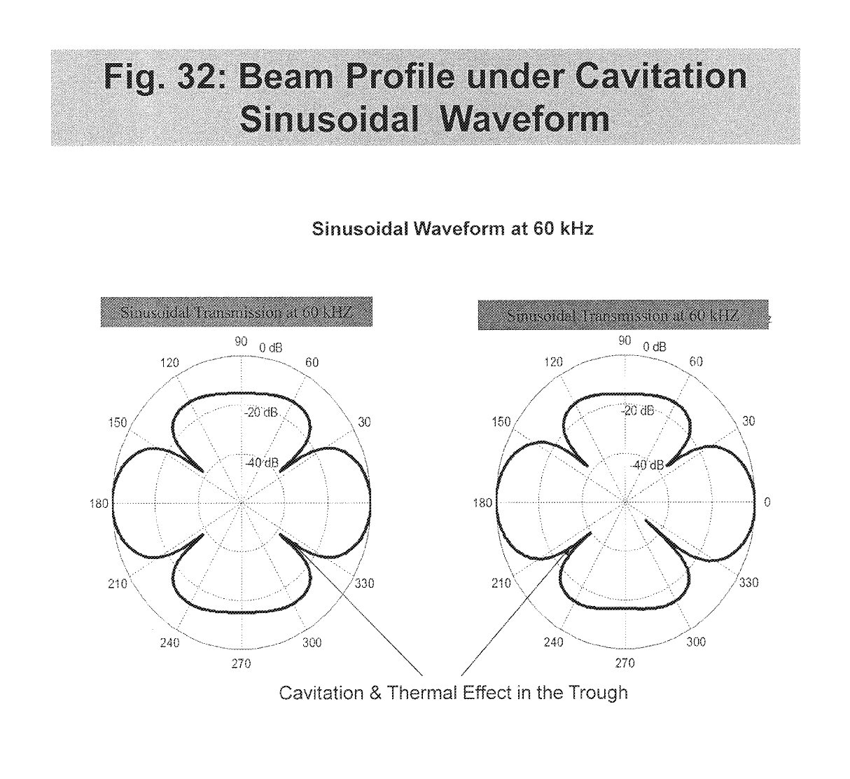

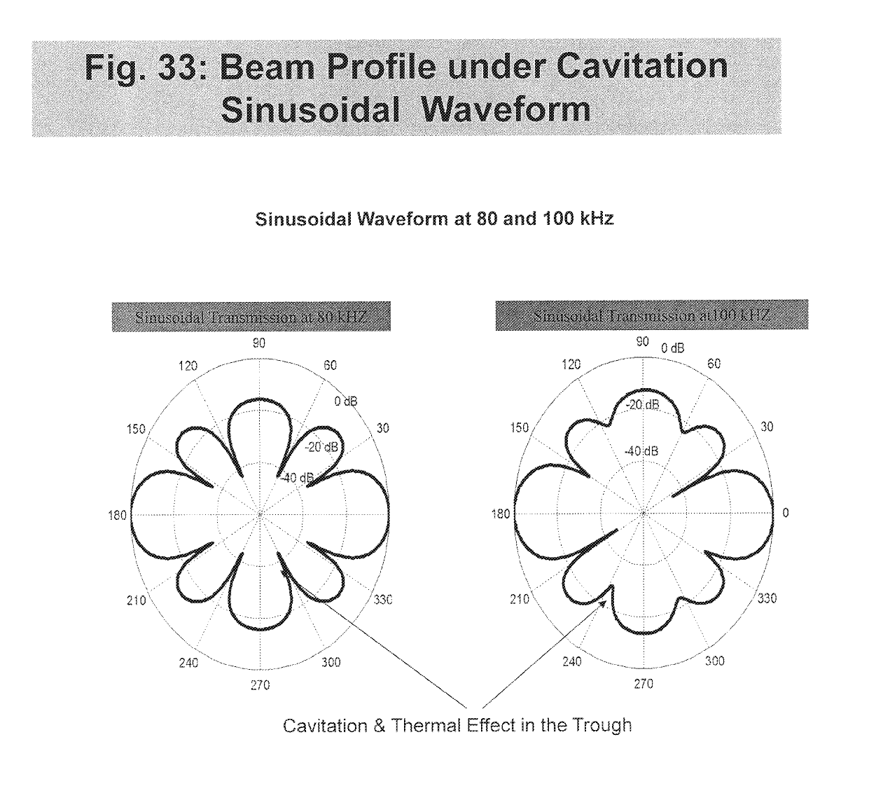

FIG. 32 and FIG. 33 illustrate the sinusoidal beam pattern with multiple cavitation spike points at 60, 80 and 100 kHz.

The beam analysis indicates that sinusoidal ultrasound, even at low frequencies, produces an irregular pattern upon a target, and in the troughs of the sonic transmission intense cavitation and thermal effects were observed.

### Apparatus Design

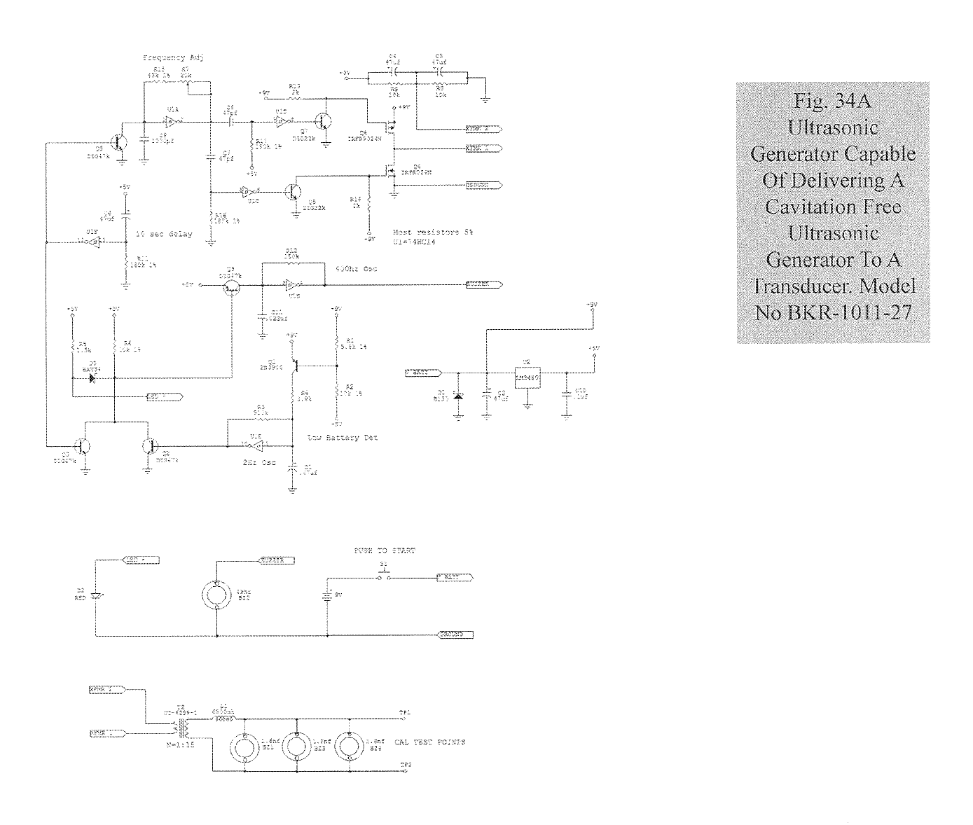

FIG. 34 is the circuit diagram of the ultrasonic generator capable of delivering a cavitation free ultrasonic generator to a transducer, Model No. BKR-1011-27, according to the present invention.

The following parts lists are for the cavitation free circuit capable of powering the special transducers at 50 msec sawtooth/50 msec square wave, at 125 mW/sq. cm intensity per transducer element in a 4-element array for a total power output of 500 mW/sq. cm, at 23-30 kHz frequency, shown in FIG. 34, according to the present invention.

The following is a parts list for the alternating ultrasound driver board for the cavitation free ultrasonic generator to a transducer shown in FIG. 34:

| CIRCUIT ID | DESCRIPTION |
|---|---|
| U1- | CD74HC14M, Hex |
| | CMOS Schmidt |
| | Trigger IC-296- |
| | 9179-5-ND |
| U2 | Quasi LDO Voltage |
| | Regulator 1C- |
| | LM34801M3- |
| | 5.0CT-ND |
| D1 | 5.6 Volt Zener |
| | Diode- |
| | MMBZ5232B- |
| | FDICT-ND |
| D3 | Schottky Diode, |
| | SOT-23-BAT54- |
| | FDICT-ND |
| Q1 | Transistor PNP- |
| | MMBT3906- |
| | FDICT-ND |
| Q2 | Pre Biased |
| | transistor NPN 22k- |
| | DDTC124TCA- |
| | FDICT-ND |
| Q3 | Pre Biased |
| | transistor NPN 22k- |
| | DDTC124TCA- |
| | FDICT-ND |
| Q4 | Power MOSFET |
| | NCHAN- |
| | IRFRO24PBFCT- |
| | ND |
| Q5 | Pre Biased |
| | transistor NPN 47k- |
| | DDTC144TCA- |
| | FDICT-ND |
| Q6 | Power MOSFET |
| | PCHAN- |
| | IRFR9O24PBFCT- |
| | ND |
| Q7 | Pre Biased |
| | transistor NPN 22k- |
| | DDTC124TCA- |
| | FDICT-ND |

| | | | |
|---|---|---|---|
| Q8 | Pre Biased | Digi-Key | 0.384 |
| | transistor NPN | Corp 1-800- | |
| | 22k- | 344-4549 | |
| | DDTC124TCA- | | |
| | FDICT-ND | | |
| Q9 | Pre Biased | Digi-Key | 0.96 |
| | transistor | Corp 1-800- | |
| | PNP 47k | 344-4549 | |
| | DDTA144TCA- | | |
| | FDICT-ND | | |
| C1 | 0.47 UT 25 v | Digi-Key | 0.0955 |
| | Tantalum A | Corp 1-800- | |
| | Electrolytic | 344-4549 | |
| | Capacitor-399- | | |
| | 3695-1-ND | | |
| C2 | 47 uf 10 v | Digi-Key | 1.3875 |
| | Tantalum B | Corp 1-800- | |
| | Electrolytic | 344-4549 | |
| | Capacitor-399- | | |
| | 3726-1-ND | | |
| C3 | 47 uf 10 v | Digi-Key | 1.3875 |
| | Tantalum B | Corp 1-800- | |
| | Electrolytic | 344-4549 | |
| | Capacitor-399- | | |
| | 3726-1-ND | | |
| C4 | 47 uf 10 v | Digi-Key | 1.3875 |
| | Tantalum B | Corp 1-800- | |
| | Electrolytic | 344-4549 | |
| | Capacitor-399- | | |
| | 3726-1-ND | | |
| C5 | | | |
| C6 | 22 uf 16 v | Digi-Key | 0.26725 |
| | Tantalum B | Corp 1-800- | |
| | Electrolytic | 344-4549 | |
| | Capacitor-399- | | |
| | 3717-1-ND | | |
| C7 | 47 pf 50 V 0805 | Digi-Key | 0.03424 |
| | Ceramic | Corp 1-800- | |
| | Capacitor NPO- | 344-4549 | |
| | PCC470CGCT- | | |
| | ND | | |
| C8 | 47 pf 50 V 0805 | Digi-Key | 0.03424 |
| | Ceramic | Corp 1-800- | |
| | Capacitor NPO- | 344-4549 | |
| | PCC470CGCT- | | |
| | ND | | |
| C9 | 1000 pf 50 V | Digi-Key | 0.2640 |
| | 0805 Ceramic | Corp 1-800- | |
| | Capacitor NPO- | 344-4549 | |
| | PCC102CGCT- | | |
| | ND | | |
| C10 | 0.1 uf 25 V 0805 | Digi-Key | 0.2295 |
| | Ceramic | Corp 1-800- | |
| | Capacitor X7R- | 344-4549 | |
| | PCC1828CT- | | |
| | ND | | |
| C11 | 0.1 uf 50 V 0805 | Digi-Key | 0.01280 |
| | Ceramic | Corp 1-800- | |
| | Capacitor X7R- | 344-4549 | |
| | PCC223BGCT- | | |
| | ND | | |
| R1 | 5.6 Ohm 5% | Digi-Key | 0.1595 |
| | 0805- | Corp 1-800- | |
| | RHM5.6kACT- | 344-4549 | |
| | ND | | |
| R2 | 3.9 Ohm 5% | Request | 0.03879 |
| | 0805- | Inventory | |
| | RHM3.9kACT- | verification | |
| | ND* | for ROHS | |
| | | compatibility | |
| R3 | 910k Ohm 5% | Request | 0.1595 |
| | 0805- | Inventory | |
| | RHM910kACT- | verification | |
| | ND* | for ROHS | |
| | | compatibility | |
| R4 | 3.9 Ohm 5% | Request | 0.03879 |
| | 0805- | Inventory | |
| | RHM3.9kACT- | verification | |
| | ND* | for ROHS | |
| | | compatibility | |
| R5 | 1.5k Ohm 5% | Request | 0.1595 |
| | 0805- | Inventory | |
| | RHM1.5kACT- | verification | |
| | ND* | for ROHS | |
| | | compatibility | |
| R6 | 10k Ohm 5% | Request | 0.1595 |
| | 0805- | Inventory | |
| | RHM10kACT- | verification | |
| | ND* | for ROHS | |
| | | compatibility | |
| R7 | 20k Ohm Trim | Request | 0.348 |
| | Pot SMD- | Inventory | |
| | P3V203CT- | verification | |
| | ND* | for ROHS | |
| | | compatibility | |
| R8 | 10k Ohm 5% | Request | 0.03858 |
| | 1206- | Inventory | |
| | RHM10kECT- | verification | |
| | ND* | for ROHS | |
| | | compatibility | |
| R9 | 10k Ohm 5% | Request | 0.03858 |
| | 1206- | Inventory | |
| | RHM10kECT- | verification | |
| | ND* | for ROHS | |
| | | compatibility | |
| R-10 | | | |
| R-11 | 510k Ohm 5% | Request | 0.01595 |
| | 0805- | Inventory | |
| | RHM510kACT- | verification | |
| | ND* | for ROHS | |
| | | compatibility | |
| R-12 | 150k Ohm 5% | Request | 0.01595 |
| | 0805- | Inventory | |
| | RHM150kACT- | verification | |
| | ND* | for ROHS | |
| | | compatibility | |
| R13 | 2.0k Ohm 5% | Request | 0.03849 |
| | 0805- | Inventory | |
| | RHM2.0kACT- | verification | |
| | ND* | for ROHS | |
| | | compatibility | |
| R14 | 2.0k Ohm 5% | Request | 0.03849 |
| | 0805- | Inventory | |
| | RHM2.0kACT- | verification | |
| | ND* | for ROHS | |
| | | compatibility | |
| R15 | 43.0k Ohm 1% | Request | 0.01827 |
| | 0805- | Inventory | |
| | RHM43.0kCCT- | verification | |
| | ND* | for ROHS | |
| | | compatibility | |
| R16 | 180k Ohm 1% | Request | 0.03654 |
| | 0805- | Inventory | |
| | RHM180kCCT- | verification | |
| | ND* | for ROHS | |
| | | compatibility | |
| R17 | 180k Ohm 1% | Request | 0.03654 |
| | 0805- | Inventory | |
| | RHM180kCCT- | verification | |
| | ND* | for ROHS | |
| | | compatibility | |
| PCB | Driver, Bare | Digi-Key | 1.43 |
| | | Corp 1-800- | |
| | | 344-4549 | |
| | Sub-Total | | 6.49641 |

The following is a parts list for the electronics used in the alternating ultrasound power board for the cavitation free ultrasonic generator to a transducer shown in FIG. 34:

| | | | EST |
|---|---|---|---|
| | | | PRICING* |
| | | | at |
| CIRCUIT ID | DESCRIPTION | SOURCE | 1,000 pcs |
| Power PCB | Bare | Digi-Key Corp 1- | 0.95 |
| | | 800-344-4549 | |
| BZ2 | 4.1 KHz Piezo-electric | Digi-Key Corp 1- | 1.392 |
| | Buzzer, Digi Key 102- | 800-344-4549 | |
| | 1115-ND | | |
| L1 | Custom 6800 uh | CET Technologies | 1.01 |
| | Inductor CT-6341 | 1-603-894-6100 | |
| T2 | Custom Transformer | CET Technologies | 2.82 |
| | CT-6299-1 | 1-603-894-6100 | |
| Test Points | Yellow, 5004K-ND | Digi-Key Corp 1- | 0.10895 |
| | | 800-344-4549 | |
| Test Points | Black, 5001K-ND | Digi-Key Corp 1- | 0.10895 |
| | | 800-344-4549 | |
| | Sub-Total | | 12.88631 |

The following is a parts list for the alternating ultrasound chassis for the cavitation free ultrasonic generator to a transducer shown in FIG. 34:

| | | | EST PRICING* at 1,000 |
|---|---|---|---|
| CIRCUIT ID | DESCRIPTION | SOURCE | pcs |
| S1 | Momentary normally | Mouser Electronics 1- | 0.99 |
| | Open Push Button, | 800-344-4539 | |
| | Mouser 10PA019 | | |
| CN1 | Battery Connector, | Digi-Key Corp 1-800- | 0.23385 |
| | Keystone, Digi-Key | 344-4549 | |
| | 2242K-ND | | |
| D2 | Red Light emitting | Digi-Key Corp 1-800- | 0.058 |
| | Diode, Digi-Key 160- | 344-4549 | |
| | 1139-ND | | |
| BZ1, BZ2, BZ3 mounted | Ultrasonic Transducer | Encapsulation Systems | |
| in array configuration on | Transducers | Bldg, 109, 1489 | |
| S.S. Plate | | Baltimore Pike, | |
| | | Springfield, PA. 19064 | |
| | | USA | |
| | | Phone: 610-543-0800 | |
| Misc-Cable | 2 inch, flat, 7 conductor, | Digi-Key Corp 1-800- | 0.25373 |
| | Digi-Key WM07A-02- | 344-4549 | |
| | ND | | |
| Misc. Washers | Lock, #2, 5 required, | McMaster Carr ph: 732- | 0.05 |
| | 91102A710 | 329-3200 | |
| Misc screws | 2-56 × ¼ plastic, 5 | McMaster Carr ph: 732- | 1.00 |
| | required, 90380A005 | 329-3200 | |
| | Sub-Total | | 15.47189 |

The device of this invention is intended to provide certain key functions:
a) Using a new transducer design and array of transducers which produce one or more differing ultrasonic waveforms can reduce or eliminate the tendency for ultrasound to generate cavitation and intense heating effects in a target material subjected to the ultrasound.
b) Using a new transducer design and array of transducers which produce one or more differing ultrasonic waveforms can provide higher power utilization efficiencies and helps to avoid the damaging effects of excessive cavitation upon the target material.
c) By varying the timing of the time present on any one waveform, when using one or more differing alternating sonic waveforms in an ultrasonic transmission cavitation formation and growth can be interrupted.
d) Further by installing a deactivation period in the timing cycle between differing alternating sonic waveforms in an ultrasonic transmission cavitation formation and growth can be interrupted.
e) A transducer design, capable of providing cavitation free ultrasound has been disclosed, in both a single element transducer and through an array of transducers, along with means or fabricating same, and making the transducer develop a mechanical waveform similar to the electronic signal delivered to the device has been disclosed.
f) The damaging effects of cavitation ultrasound have been demonstrated in drug interactions whereupon Lispro insulin was found to degrade with conventional single waveform sonic energy, sinusoidal ultrasound. Beam profiles of conventional ultrasound exhibit irregular shaped transmission energy, with intense thermal effects within the sonic patter, but not with a patterns discovered through the use of alternating ultrasonic waveform transmissions.

Having described the invention in the above detail, those skilled in the art will recognize that there are a number of variations to the design and functionality for the device, but such variations of the design and functionality are intended to fall within the present disclosure.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I claim:

1. A method of providing cavitation free ultrasound in an ultrasonic device, whereby an ultrasonic signal employs a combination of two or more waveforms, and whereby the growth of the acoustic signal is interrupted from becoming cavitational.

2. The method of claim 1, wherein a first waveform is a sawtooth waveform followed by a second, square waveform.

3. The method of claim 1, wherein the ultrasound is applied continuously.

4. The method of claim 1, wherein the ultrasound is applied pulsed.

5. The method of claim 1, wherein the ultrasound has a frequency greater than 20 kHz.

6. The method of claim 1, wherein timing available to each waveform is kept below 400 milliseconds.

7. The method of claim 6, wherein the timing that each waveform is present is varied from any combination of time under 400 milliseconds.

8. The method of claim 7, wherein the ultrasound timing is varied from about 50 milliseconds to about 90 milliseconds for the first waveform and from about 10 milliseconds to about 50 milliseconds for the second waveform.

9. The method of claim 6, wherein the timing available to each waveform is kept below 400 milliseconds, followed by a deactivation period for a sufficient period of time again to avoid the formation of cavitation, and then followed by re-cycling of alternating sonic transmission, in series.

10. A transducer which is capable of delivering mechanically a waveform fed to it electronically from a first waveform to any other second waveform, wherein the waveforms are any one of a sine waveform, a sawtooth waveform, a square waveform and a triangular waveform.

11. A transducer as claimed in claim 10, which is constructed using a piezoelectric or magneto restrictive crystal at the center, surround by an air gap above and below the crystal, encased in aluminum foil at its bottom and titanium foil at its top, attached to rubber gaskets which allow the sonic energy emanating from the crystal to propagate in one direction, toward a target, with reduced wasted ultrasonic signal, thereby enabling a greater sonic conversion efficiency from the mechanical sonic device and to enable the transducer to deliver a mechanical waveform matching the electrical waveform delivered to it by an electronic control circuit.

12. A transducer which is capable of delivering cavitation free ultrasound, which employs a reflector on a top face of the transducer to reflect ultrasonic signals back to a target.

13. A transducer which is capable of delivering cavitation free ultrasound, which employs one or more individual transducer discs or elements in an array, placed over a stainless steel face plate, and which cause the face plate to irradiate harmonic ultrasound in resonance to the ultrasound delivered from the transducer discs affixed to it, wherein the face plate and transducer disc array are covered by a block containing a flexible foam rubber layer between the stainless steel face plate and the block housing, whereby increasing overall intensity of the transducer and increasing the diameter of surface area to the overall sonic transmission.

14. A method of delivering cavitation free ultrasound, which produces a sonic pattern upon a target, which is spherical and which avoids troughs in the beam profile, thereby avoiding cavitation effects upon the target material subjected to the ultrasound transmission.

15. A method of delivering cavitation free ultrasound, which employs one or more alternating sonic waveforms where one waveform is a triangular wave front where the frequency and amplitude of the wave front is diminishing over time, thereby preventing the growth of a cavitation or thermal effect to the ultrasound transmission.
